(19) United States
(12) Patent Application Publication
(10) Pub. No.: US 2023/0355898 A1
(43) Pub. Date: Nov. 9, 2023

(54) NEBULIZER AND NEBULIZED ANTI-VIRALS

(71) Applicant: MICRONIZATION TECHNOLOGIES AND THERAPEUTICS GROUP LLC, Gulfport, MS (US)

(72) Inventors: Andrey FACTOR, Farmington Hills, MI (US); Gary Robert KELM, Cincinnati, OH (US); Edward Ronald GRIFFOR, Grosse Point Park, MI (US); Donald O. KING, Corinth, MS (US)

(21) Appl. No.: 18/245,968

(22) PCT Filed: Sep. 21, 2021
(86) PCT No.: PCT/US2021/051210

§ 371 Date: Mar. 20, 2023

Related U.S. Application Data
(60) Provisional application No. 63/081,414, filed on Sep. 22, 2020.
(60) Provisional application No. 63/244,548, filed on Sep. 15, 2021.
(60) Provisional application No. 63/246,150, filed on Sep. 20, 2021.

(51) Int. Cl. A61M 11/02; A61K 31/4706; A61K 9/00; A61K 47/10; A61K 9/107; A61K 47/22; A61M 15/00

(52) U.S. Cl. CPC A61M 11/02; A61K 31/4706; A61K 9/0078; A61K 47/10; A61K 9/1075; A61K 47/22; A61M 15/002; A61M 15/0021

(57) ABSTRACT

An antiviral medication which may be an aerosolizable formulation having a pharmaceutically active anti-viral compound present as a neutral compound selected from the group of free acid, free base, water insoluble salt and water insoluble ion pair); an excipient capable of forming a liquid complex with the pharmaceutically active anti-viral compound; and a polymeric surfactant suitable for pulmonary administration. A nebulizer to deliver the antiviral medication may have a compressed air chamber in communication with a medication chamber, the communication sealed by a spring valve in a rested state. A nebulizer chamber is in communication with the medication chamber, the nebulizer chamber having a pressure release orifice to deliver a stream the nebulized particle stream to the facemask and a continuously variable nebulizing pressure feed at the pressure release orifice.

### CROSS-REFERENCE TO RELATED APPLICATIONS

This application is a U.S. national phase application filed under 35 U.S.C. § 371 of International Application No. PCT/EP2021/051210, filed Sep. 21, 2021, designating the United States, which claims priority from U.S. provisional application 63/081,414 filed Sep. 22, 2020; U.S. provisional application 63/244,548 filed Sep. 15, 2021; and U.S. provisional application 63/246,150 filed Sep. 20, 2021, which are hereby incorporated herein by reference in their entirety for all purposes.

### FIELD

The present invention relates to nebulized anti-viral medications and nebulizers for delivering medication, and more particularly disposable nebulizers having onboard compressed gas to aerosolize the medication and to provide continuously variable droplet size of the medications.

### BACKGROUND

A nebulizer is a drug delivery device that is used to deliver medication in the form of an aerosolized mist into the lungs of a patient. Nebulizers use oxygen, compressed air, ultrasonic power, and the like to break up solutions and suspensions into small aerosol droplets that are inhaled from the mouthpiece of the device. Nebulizers are commonly used for the treatment of respiratory diseases, such as asthma, cystic fibrosis, and COPD. Due to the Covid-19 pandemic, there is increased interest in and need for improvements to nebulizers.

The most commonly used nebulizers are jet nebulizers. Jet nebulizers are connected by tubing to a supply of a propellant, such as compressed gas, such as air or oxygen. Upon release into the nebulizer, the compressed gas flows at high velocity through a liquid medicine to turn it into an aerosol that is inhaled by the patient.

Conventional nebulizers, while effective in delivering medical treatment to the lungs, suffer from drawbacks. A nebulizer is expensive. Nebulizers are also bulky and are generally used at a single location, such as in a hospital room or a home. While some nebulizers are smaller and can be carried by hand, the units are not suitable for convenient everyday use or for distribution to remote areas. It would be advantageous to have a device that matches the functionality of a conventional nebulizer, but which can be carried in a pocket, a purse, or the like.

Conventional nebulizers operate from 13 psi all the way up to 50 psi. A jet nebulizer with a lower flow or pressure will increase particle size and a higher flow or pressure will decrease particle size. Therefore, using higher air flow rate in nebulizer therapy could decrease the amount of treatment time needed to deliver the set amount of medication as well as a decrease in particle size.

Aerosolized drugs can also be delivered by hand-held devices known as metered dose inhalers. Metered dose inhalers are typically used to deliver multiple metered doses on an as-needed basis, such as for asthma. Metered dose inhalers operate differently from the present invention.

Potential pulmonary delivery of antiviral drugs is mentioned in the art. Pulmonary delivery of hydroxychloroquine has been described in US Pub 2008/0138397 A1 to Schuster et al., which describes the use of a sustained release formulation of hydroxychloroquine that minimizes the bitter or otherwise unpleasant taste of a drug or its potential to stimulate the cough reflex when administered by the pulmonary/inhalation route. A liposomal formulation of hydroxychloroquine is specified. Liposomal formulations are sensitive to shear and may be compromised by the process of aerosolization required for the targeted delivery. U.S. Pat. No. 5,384,128 to Meezan, et al. generally discloses increasing the permeability of epithelial cells to chloride ion as means of treating cystic fibrosis.

Despite advances in the art, there remains a desire and a need to improve nebulizers and the bioavailability of drugs administered at the respiratory tract of an animal/human.

### SUMMARY

It is an object of the inventions to provide disposable nebulizers.

It is an object of the inventions to provide to disposable nebulizers that can be pre-loaded with medicine.

Another object of the inventions is to provide disposable nebulizers that are configured for one-time use by individual users prior to disposal.

The foregoing objectives are achieved by providing a disposable nebulizer having the features described herein.

According to one approach, a nebulizer is provided suitable for medication delivery to the lungs, having a compressed air chamber in communication with a medication chamber, the communication sealed by a spring valve in a rested state, the spring valve being openable in an actuated state, a nebulizer chamber in communication with the medication chamber, the nebulizer chamber having a pressure release orifice, a facemask integral or in direct communication with the nebulizer chamber, wherein the nebulizer chamber is configured to deliver a stream the nebulized particle stream to the facemask, wherein the compressed air chamber is configured to have the volume and pressure of air needed to nebulize the medication through the pressure release orifice, wherein the nebulizer chamber has a continuously variable nebulizing pressure feed at the pressure release orifice; and wherein the spring valve is released by a pair of levers that when actuated, force the medication chamber and the gas chamber together to open the spring valve.

In one approach, the continuously variable nebulizing pressure feed has a retractable and extendable needle to retract and extend into the pressure release orifice.

The present nebulizer can have the retractable needle attached to a threaded shaft disposed within a threaded bore, which is rotatable by a control knob to retract and extend into the pressure release orifice. The nebulizer chamber can be configured to produce a nebulized particle stream in the range of 1-10 μm.

In one approach, the medication can be an antiviral with a carrier, which is nebulizable to a particle stream in the range of 3-5 μm.

The nebulizer can be configured so that the force required to open the spring valve to an actuated state is less than 3 nM of force.

The nebulizer can be configured so that the actuation levers remain aligned along its travel path by a guide track on one of the levers and a paul, which is guided within the track, on the other lever and that the compressed air chamber is configured to hold up to 120 PSI of air, preferably in the range of 20-60 PSI.

The nebulizer has a pressure release orifice and can be configured to produce aerosolized fluid of varying droplet/particle sizes ranging from 1-10 micrometers in diameter, preferably 3-5 micrometers. The pressure release orifice can be 0.35-2.0 mm in diameter.

Accordingly, to advance at least the aforementioned deficiencies in the art, described herein are compositions and methods related to site-specific delivery of a pharmaceutically active compound to the respiratory tract of an animal/human, and in particular compositions and methods related to delivery of an approved pharmaceutically active compound (“drug”) with antiviral activity to a viral infection site of the respiratory tract which synergistically maximizes interaction of the drug with extracellular virus particles, inhibits/reduces viral epithelial cell entry through potential interaction with sites of extracellular viral binding to epithelial cell membranes, and potentiates drug partitioning into epithelial cells.

According to one approach, an inhalation formulation is provided having an aerosolizable formulation (such as an aqueous aerosolizable formulation) and may have a pharmaceutically active anti-viral compound present as a neutral compound (free acid or free base, or water insoluble salt or ion pair); an excipient capable of forming a liquid complex with the pharmaceutically active anti-viral compound; and a polymeric surfactant suitable for pulmonary administration. In one approach the pharmaceutically active anti-viral compound is an aminoquinoline. In another approach, the pharmaceutically active anti-viral compound is at least one of chloroquine (CQ), hydroxychloroquine (HCQ) and amodiaquine.

The excipient forming a liquid complex with the pharmaceutically active anti-viral compound can be present in a 0.2:1 to a 5:1 excipient to drug mass ratio. The excipient forming a liquid complex with the pharmaceutically active anti-viral compound may be present in a 1:1 excipient to drug mass ratio. The excipient may be propylene glycol, USP.

The polymeric surfactant suitable for administration to the respiratory tract may be capable of producing a micellar solution with the drug liquid complex at mass ratios to the drug liquid complex of 8:1 or lower.

The polymeric surfactant suitable for administration to the respiratory tract is capable of producing a micellar solution with the drug liquid complex at mass ratios to the drug liquid complex of 4:1. The polymeric surfactant suitable for administration to the respiratory tract is capable of producing a micellar solution with the drug liquid complex is tocopheryl polyglycerol succinate. The aerosolized formulation may comprise micelle sizes less than 100 nm. According to another approach aerosolized aqueous formulation may comprise micelle sizes less than 50 nm.

The aerosolized droplets may be less than 5 μm and lipoidal particles containing approved drugs are less than 100 nm.

The pharmaceutically active anti-viral compound may be a non-charged chloroquine in a 1:1 ratio with the excipient that is lipophilic and liquid at body temperature.

In other various embodiments, the eutectic excipient may be USP propylene glycol; the formulation may be an isotonic micellar solution; the aerosolized droplets may be 1-10 μm, 1-5 μm, 5-10 μm, 3-5 μm; the formulation eutectic excipient has a lower melting point than the drug.

In another approach, an aminoquinoline eutectic formulation is provided having the drug free base, a physiologically compatible eutectic excipient, in a 1:1 mass ratio, that is liquid at body, and/or ambient temperature, a polymeric surfactant suitable for administration to the respiratory tract of an animal/human that is capable of producing a micellar solution of the aminoquinoline eutectic system at surfactant:eutectic system mass ratio of 4:1 or lower and having micelle sizes less than 100 nm and ideally less than 50 nm, and an isotonic aqueous vehicle at physiological pH. In this approach, the excipient is hydrophilic and selected from the group of: tetraethylene glycol, propylene glycol USP, 1,3-propanediol, 1,3-butylene glycol, pentylene glycol, and combinations thereof. The polymeric surfactant may be D-a-tocopheryl polyethylene glycol 1000 succinate NF (TPGS). The aerosolizable formulation may have a viscosity of +/−0.10 cP of 1.92 cP. The aerosolizable formulation may be configured for presence of the antiviral's active ingredients in optimized quality and minimal quantity in vivo for a duration in those regions of 1 day to 30 days.

The aerosolizable formulations herein can be shelf stable at ambient temperatures between 60 and 75 degrees Fahrenheit (15 and 25 degrees Celsius) for up to 6 months.

A nebulizer is provided to aerosolize the present formulations having a mechanism to continuously adjust aerosolized droplets size in the range of 1-10 μm.

Other features will become more apparent to persons having ordinary skill in the art to which the assemblies pertain and from the following description and claims. While the features described herein may be susceptible to various modifications and alternative forms, specific embodiments thereof are herein described in detail. It should be understood, however, that the detailed description thereto are not intended to be limiting to the particular form disclosed, but on the contrary, the intention is to cover all modifications, equivalents and alternatives falling within the spirit and scope of the subject matter as defined by at least the appended claims.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 is a front-side perspective view of one preferred embodiment of a disposable nebulizer of the invention.

FIG. 2 illustrates a perspective front view of a device in accordance with another one of the present embodiments;

FIG. 3 illustrates a side cutaway view of the actuation arms in an open position;

FIG. 4 illustrates a side cutaway view of the actuation arms in a closed position;

FIG. 5 illustrates a rear elevational view of the device of FIG. 2;

FIG. 6 illustrates a front elevational view of the device of FIG. 2;

FIG. 7 illustrates a right side elevational view of the device of FIG. 2, the left side elevational view being a mirror image;

FIG. 8 illustrates a right side cut-away elevational view of the device of FIG. 2 taken along section lines VIII-VIII in FIG. 13;

FIG. 9A illustrates a close-up right side cut-away elevational view of the device of FIG. 2 at portion IX-X of FIG. 8 with the jet needle advanced into the jet housing orifice;

FIG. 9B illustrates a close-up right side cut-away elevational view of the device of FIG. 2 at portion IX-X of FIG. 8 with the jet needle retracted from advanced into the jet housing orifice;

FIG. 10 illustrates a close-up right side cut-away elevational view of the device of FIG. 2 at portion IX-X of FIG. 8 while in use;

FIG. 11 illustrates a perspective rear view of a device in accordance with FIG. 2;

FIG. 12 illustrates an exploded perspective front view of a device of FIG. 2;

FIG. 13 illustrates a top view of the device of FIG. 2; and

FIG. 14 illustrates a bottom view of the device of FIG. 2.

FIG. 15 shows a schematic of a human respiratory anatomy.

While the features described herein may be susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by at least the appended claims.

### PREFERRED EMBODIMENTS OF THE INVENTION

In the following detailed description of the preferred embodiments, reference is made to the accompanying drawings which form a part hereof, and in which are shown by way of illustration specific embodiments in which the invention may be practiced. It is to be understood that other embodiments may be utilized and structural changes may be made without departing from the scope of the present invention.

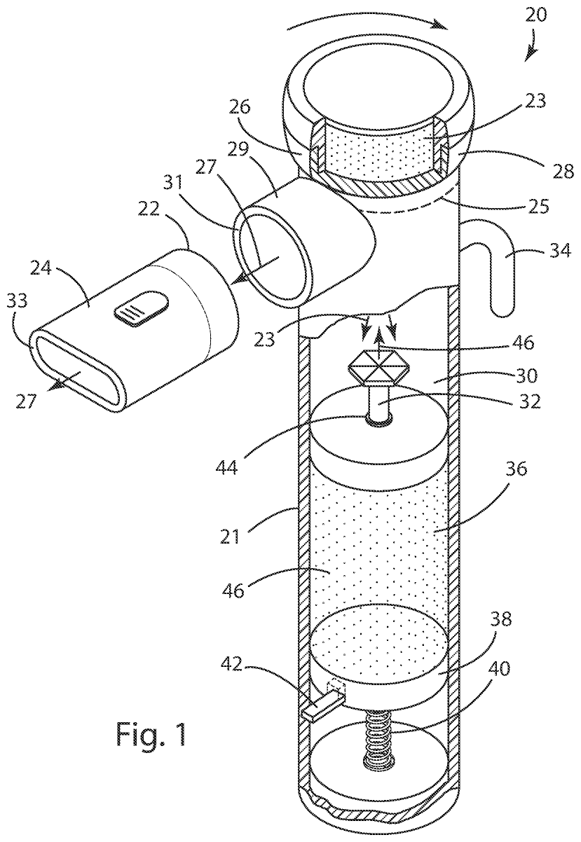

FIG. 1 shows an exemplary disposable nebulizer 20 of the present embodiments comprising generally a main body housing 21, a pre-loaded pressure delivery member 40, such as a spring, on a lower end, an adjacent air chamber 36 above the pressure delivery member 40, a nebulization chamber above the air chamber, and a storage chamber 28 on or adjacent a top end. The storage chamber is configured to store a single dose of medication 23. The storage chamber is also configured for selective release of the medication from the storage chamber into the nebulization chamber. In the embodiment of FIG. 1, release of the medication is achieved by turning the storage chamber with a twist valve 26 to break a twist valve chamber seal 25. A pressure relief valve 44 (like a tea kettle, for example) is positioned between the air chamber 36 and the nebulization chamber 30.

A jet apparatus 32 may be provided above and in communication with the pressure relief valve 44. The jet apparatus is configured to aerosolize the medications when they are released from the medication storage chamber 28 into the nebulization chamber 30. When the preloaded delivery member 40 (shown as a spring as an example) is released by a user, using the lock lever/sear 42 for example, the air 46 in the air chamber 36 is compressed and evacuated through the pressure relief valve 44, which opens at a certain pressure. The compressed air 46 is further compressed by the jet apparatus 32 prior to being expelled into the nebulizer chamber 30, where it aerosolizes or nebulizes the medicine 23 to form the aerosolized/medication 27.

In the embodiment of FIG. 1, the nebulization chamber 30 is an L-shaped tube including an upright chamber and a side pipe 29 for delivery of nebulized medicine 27 via a nebulized exit point 31. A mouthpiece 24 is attached to the side pipe 29. The mouthpiece 24 includes an input opening 22 opposite a nebulizer opening 31 and an output opening 33. The user puts his or her mouth on the mouthpiece 24 to inhale nebulized medicine 27.

A handle, such as the side handle 34 shown in FIG. 1, can be provided to assist a patient/user in holding the device.

In embodiments, the disposable nebulizer 20 is a single use nebulizer unit that does not require a power source. In the embodiment of FIG. 1, the pre-loaded discharge mechanism 40 is configured like a spring-piston air gun (also known as a spring gun or simply a “springer”) that operates by means of a spring-loaded piston pump assembly 40 contained within the air compression chamber 36. The spring 40 and piston 38 are housed in a separate chamber from the nebulization chamber 30 where the medications are aerosolized.

In the embodiment of FIG. 1, the pre-loaded pressure delivery member 40 incorporates discharge mechanisms similar to ones found in air guns. Traditionally, in air guns, a grease-lubricated steel coil spring is used as the powerplant main spring. However, unlike a fire arm where the user needs to manually cock the gun by flexing a lever connected to the pump assembly, which pulls the pump piston rearwards and compresses the main spring until the rear of the piston engages the sear, the nebulizer unit 20 of the present embodiments comes already cocked with the sear 42 in place, retaining the piston 38 and spring 40 in a loaded configuration. In order to fire the pressure delivery member, the user tums a lever or knob at the bottom of the handle to disengage the sear 42. This allows the main spring 40 to decompress and release its stored elastic potential energy, pushing the piston 38 forward and thereby compressing the air 46 in the pump cylinder, air chamber 36. Compression of air 46 in the pump cylinder/air chamber 36 triggers the pressure release valve 44 once the air pressure has risen enough to overcome the static friction and propel air 46 forward up into the nebulizer chamber 30 where the medication 23 is aerosolized by the expanding column of pressurized air in air chamber 36. The device 20 distributes the medication into a fog/mist 27 that is inhaled by patients. In embodiments, the medication 23 is pre-loaded in the upper medication chamber/top 28. The top 28 may be configured to twist, which would open the chamber 28 where the medication 23 would then drop down into the nebulization chamber 30. Once the medicine 23 has been released into the nebulization chamber 30, the lever 42 or knob is turned to trigger air flow, disengage the sear and begin nebulizer treatment.

It is theorized that the use of higher air flow will improve the efficacy of the device for nebulizer therapy. Using higher air flow rate may decrease the amount of treatment time needed to deliver the set amount of medication as well as a decrease in particle size.

The circumference and/or length of the air chamber can be adjusted to allow the necessary ambient air pressure volume needed before compression begins.

The spring 40 pressure and the jet apparatus/regulator 32 can be adjusted for specific PSI requirements.

The flow line diameter after the pressure relief value 44 can be adjusted to achieve the required CFM (cubic feet of air moved).

The piston assembly can be substituted with a pre-charged pneumatic cylinder connected to the pressure relief valve 44 or the cylinder itself can be made of sufficient material to allow it to serve as its own pre-charged pneumatic chamber which would discharge through the pressure relief value.

The storage chamber 28 for the pre-filled medication 23 can be outfitted with a mechanical fill slot designed for high speed or automated filling machines.

The storage chamber 28 can alternatively be outfitted with a cap intended to allow manual or mechanical filling with the cap installed after the storage chamber has been filled.

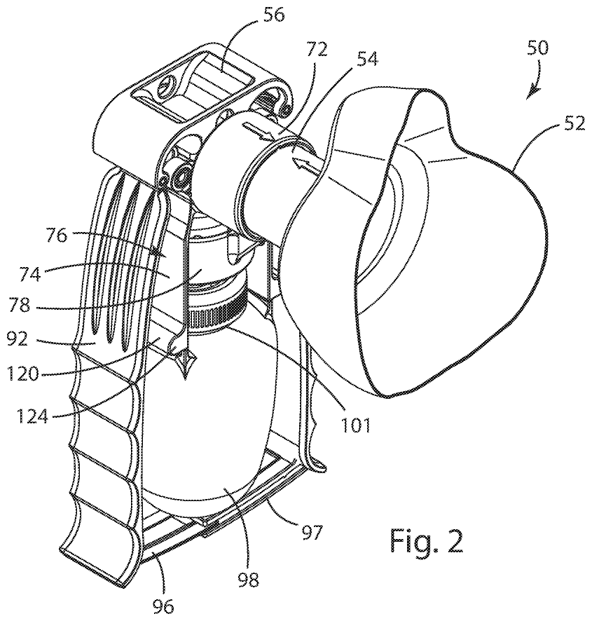

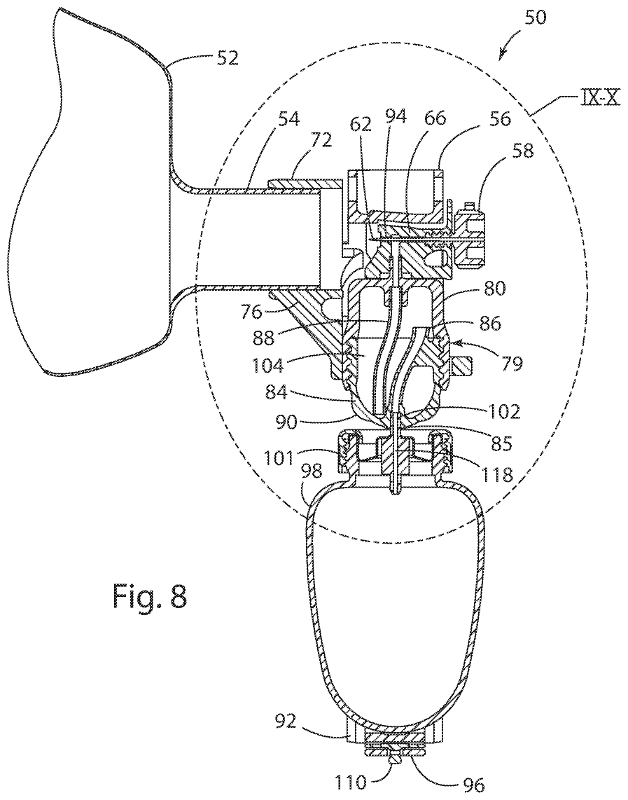

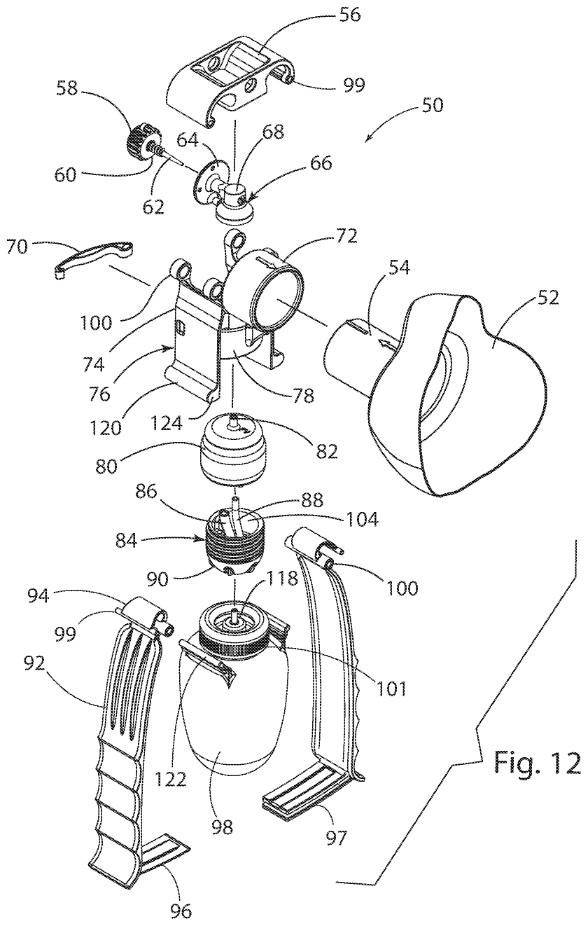

FIGS. 2-14 show an alternate approach to the present embodiments. In this approach, onboard compressed air is used, which can be deployed using actuation arms to activate living hinges to release compressed air to force medication through the nebulizer. Also, the nebulizer portion is adjustable to produce variation in droplet size of the nebulized medicine. The change in droplet size allows the option deeper lung penetration as the droplet size decreases. This solution mediates the cause of infection and is substantially delivered to targeted tissue, avoiding the adverse effects of medications on healthy tissue.

The onboard compressed air provides the force required to aerosolize a suitable fluid, including fluid-based pharmaceuticals, thus eliminating the need for user access to electrical or other power sources. The adjustable nozzle diameter allows the nebulizer to produce aerosolized fluid of varying droplet/particle sizes, ranging from, for example, 1-10 micrometers in diameter (preferably about 3-5 micrometers, and most preferred at about 3 micrometers).

These two features can allow the nebulizer device to achieve longer treatment durations, by increasing the pressure in the compressed air chamber and/or by decreasing the droplet size by decreasing the nozzle diameter using the adjustable nozzle. The targeted delivering of more precise size droplets is advantageous to target and treat specific areas of tissues within the respiratory tract of a user. For example, this feature allows medication to target the upper or lower respiratory tract with custom dosing levels that can be specified based on the stage of exposure Additionally, the lever geometry of the actuation arms minimizes the force required for actuation to less than 3 nM of force; a force that has been shown to be achievable by the 95th percentile of human subjects.

This embodiment may be manufactured using additive manufacturing methodologies such as 3D printing and crimping technologies. Limiting the complexity to additive materials and a small number of generally available valve components reduces supply chain vulnerabilities. Ease of manufacture of the present embodiments can be realized because: it can be 90 percent 3D printed, design changes are easier than injection molded devices, uniform materials are used, low work op count (about 3 work operations), low effort to integrate with other equipment, shipping and drug design (modify jet for different fluid specifications), easy modeling and design advancement, and easy to prepackage.

The device uses a small number of components/parts, which renders the manufacture and sourcing less vulnerable to disruption and has less failure modes. There are reduced key components in the present embodiment compared to 50 or more parts in nebulizers known in the art. Since most of the components of the present embodiment can be 3D printed, this provides 2 critical pandemic solutions: (a) the design can be modified and improved with changes to a software model and easily distributed, thus allowing access to a vast pool of intellectual capital worldwide and rapid sharing and testing of prototypes, and (b) the potential to utilize a vast network of micro-manufacturing facilities worldwide. This is a significant step forward in eliminating supply chain disruptions when it is critical to produce and deliver lifesaving medications in a timely manner anywhere in the world. Another advantage is that the present embodiments are small in size from the perspective of manufacture, logistics and useability.

In this embodiment, no external power is needed (e.g., neither electrical grid connectivity nor battery are required) and no fuels delivered to a site of use. This embodiment is propelled only by elastic or potential energy (spring piston or pre-charged pneumatic compression). It therefore does not require external delivery of fuel or electricity, thus not dependent on variations in the local specification for electoral current or its availability. This allows simple administration to a common design, which can access more points of manufacturing and provide a more rapid response chain for the ultimate delivery of potentially life-saving medications anywhere in the world.

Thus, the present embodiment provides a disposable—one-time use—unit dose delivery system. The device provides a valuable clinical application and a key advancement in allowing the preferred results of direct supply to the pulmonary tract of micronized particles via nebulization, while avoiding the risk of traditional nebulizers. For example, there is no recirculated air risk. The unit can be used by the patient without requiring close-in assistance or exposure to exhaled droplets. The unit is disposable and poses no need to clean between applications. In total this provides both key solutions to enable nebulized delivery of medications and a more sanitary environment to help reduce the transmission and spread of respiratory infections during and after treatment. The present embodiments ensure that ambient/‘fresh’ air can be drawn into the atomized medication flued on inhale and that ‘used’ or ‘exhaust’ air can escape from the mask.

The functional and ergonomic design of the present embodiments is engineered to use readily available medical grade polymers with most of the parts made from a single material. This helps ensure drug efficacy and enhanced sterility for extended periods of time. It also promotes supply chain integrity by avoiding multiple materials sourcing for final manufacture and assembly.

The present embodiments are easy to use in the absence of any assistance thus reducing exposing others to infection. The device is reliable since no independent of energy source is needed resulting in less failure modes.

The present embodiments use “green” and socio-economically positive technology. By-products of manufacturing process are fully re-useable; power independence means reduction of producing toxic materials associated with compressor or batteries; manufacturing power and pollution footprint minimum; and an ability to penetrate into underserved markets with limited resources.

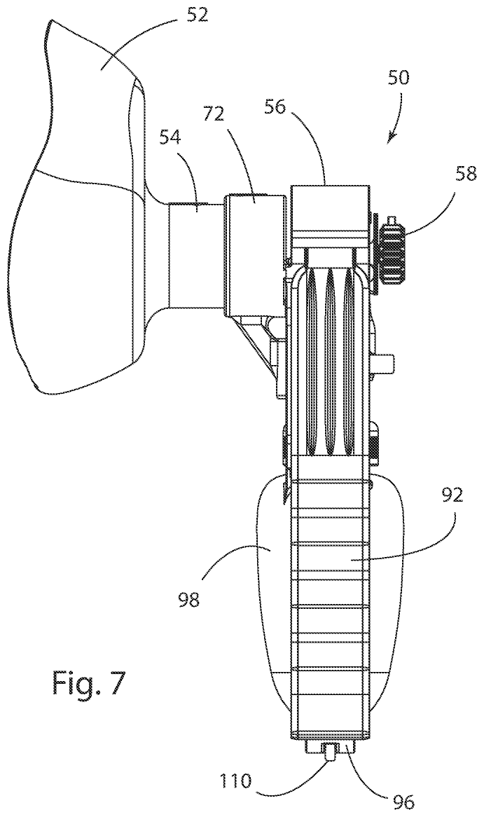

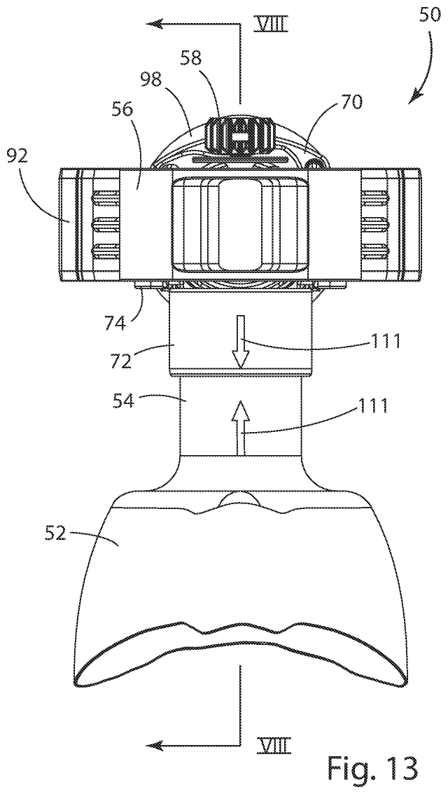

As shown in the figures, a portable nebulizer 50 of the present embodiments has a facemask 52 having an aerosol chamber 54 sized to mount to a face mask/housing interface 72. As a guide, alignment arrows 111 (FIG. 13) may be used to ensure the facemask 52 is properly installed and aligned. Nebulizer 50 has a compressed air/gas chamber 98 mounted to a nebulizer housing frame 76 by fins 122 configured to slidingly engage grooves 120 until it reaches stop 124 (FIG. 6). A medication chamber 79 an has a lower medication chamber 84 and an upper medication chamber cap 80; compressed air chamber interface 85 and an upper medication chamber interface to nebulizer frame 82. A connector 87 connects lower medication chamber 84 and upper medication chamber cap 80. Connector 87 can be a threaded mount, a snap mount, and the like. Alternative, upper medication chamber 79 may be formed of a single piece and preloaded with medication during production. Lower medication chamber 84 may have ergonomic indentations 90 to assist in unscrewing a threaded connector 87 to facilitate filling the medication chamber.

Medication chamber 79 and compressed air/gas chamber 98 are connected by a lower medication chamber compressed air chamber interface 85. Medication chamber 79 is held in place not only by interface 85, but also by the nebulizer frame 76 having living hinge/frame interlaces 74 and a latch arm lock 70 attached to the living hinge/frame interlaces 74 and a stop 78. Interface 85 has a spring-loaded valve 118 having a hollow stem 102 to insert to a medication chamber 79 bore 126 configured to receive stem 102. Valve spring 118 is tensioned to seal compressed air chamber 98 in its rested state and to release the compressed air in its actuated state. As shown, medication chamber 79 has a compressed air channel 86 to receive the compressed air in the actuated state from stem 102. Air channel 86 may be configured to extend above the medication 104 already present in medication chamber 79, though not required. As air is forced into the medication chamber 79 in the activated state, pressure is placed on medication 104 to force it through aerosol channel 88 and into the nebulizer. In one embodiment, 3 ml of medication can be placed in a medication chamber 79 having a volume of 5 ml. The compressed air chamber 98 is configured per application to deliver enough pressure to deliver the medication through the pressure release orifice.

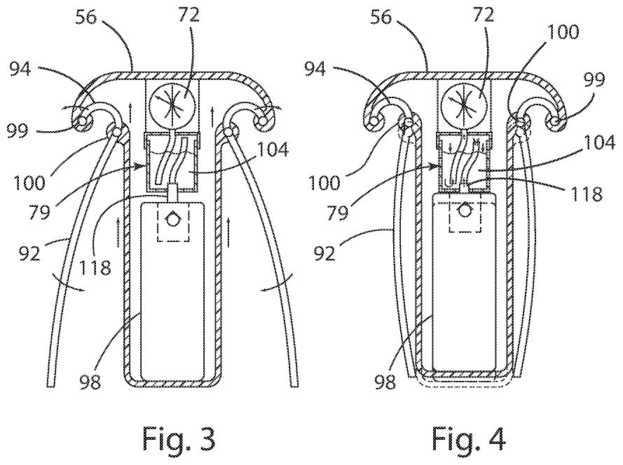

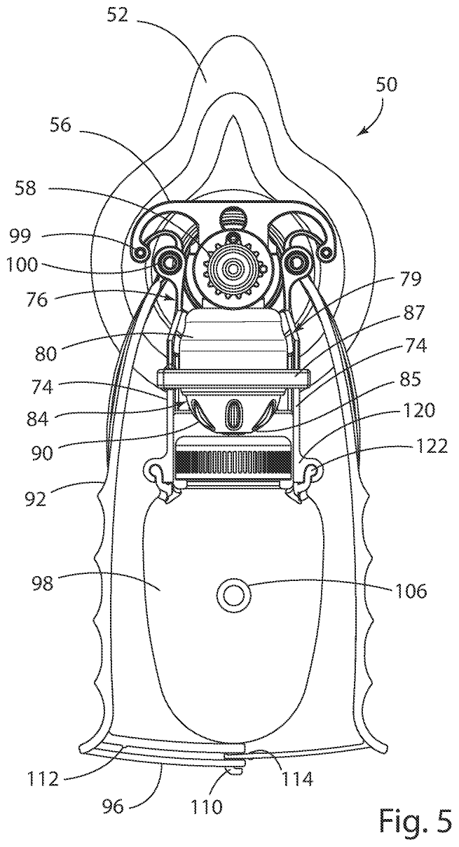

FIGS. 3 and 4, provide a simplified example of the activation step to release the air from compressed air chamber 98. As shown, a pair of actuation arms 92 are mounted to compressed air chamber 98 at its base and on the nebulizer frame 76 in FIGS. 2, 5-14.

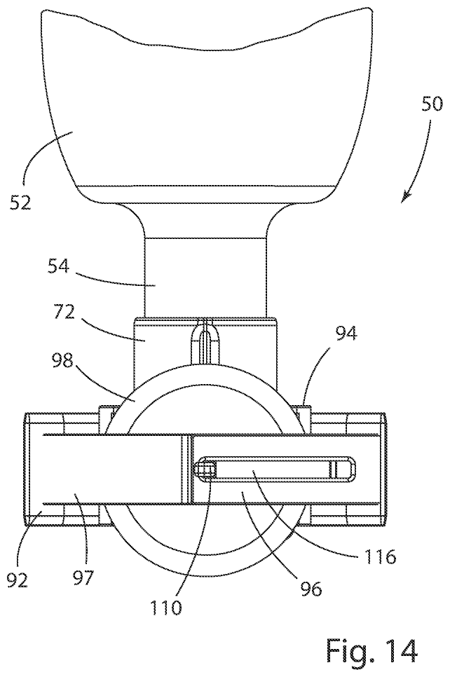

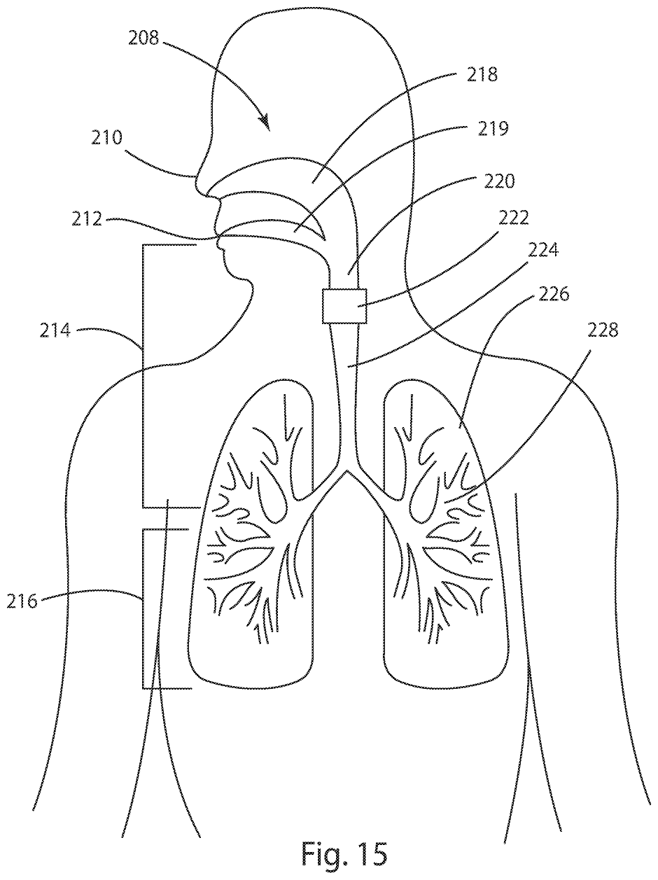

Actuation arms 92 are also mounted as a living hinge at hinge points 99 and 100. Hinge point 100 is mounted to nebulizer frame 76 and hinge 99 is mounted to a living hinge/nebulizer frame interface 56. Pivot points 99 and 100 are configured to allow activation of the release of air from the air chamber 98 using less than 3 nM of force. As shown, in the at rest state, actuation arms 92 extend away from the device. To keep the activation arms from extending too far or to move out of alignment when activating the air chamber, an actuation arm lock 6 may be provided having wings 96 and 97 held in place by a paul 110 guided along a predetermined length of travel by track 116. Thus, the present embodiments ensure that the ‘wings’ 96 and 97 on the bottom of the two nebulizer handles 92 remain aligned along the path of actuation motion and that they not be able to disengage due to paul 110 guided in a track 116 (FIG. 14). Once actuation arms 92 are fully closed onto the device, opposing ribs 112 and 114 provide a means to hold the arms closed. Rib 114 is moved along its track until it is forced past rib 112 by the user. Once this has occurred, rib 112 can hold rib 114 from allowing the actuation arms to open again.

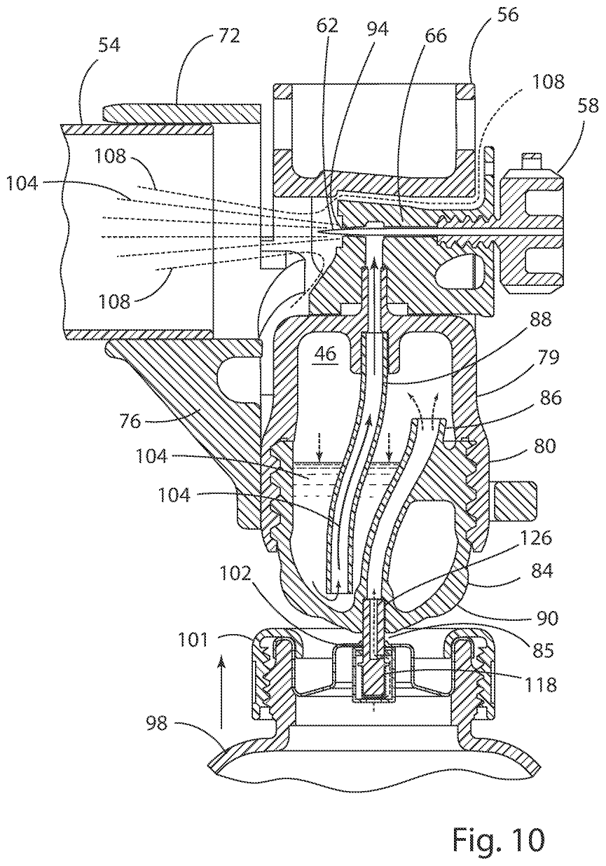

As the actuation arms 92 are squeezed to rotate them toward the device the compressed air chamber 98 and nebulizer frame 76 are forced against the living hinge/nebulizer frame interface 56. Once they meet, the user needs to add the additional force needed to continue travel of the actuation arms 92 to squeeze the interface of the air chamber 98 and nebulizer frame 76 forcing spring valve 118 downward, thus opening valve 118 and forcing air into the medication chamber 79 (See, FIG. 4 and FIG. 10).

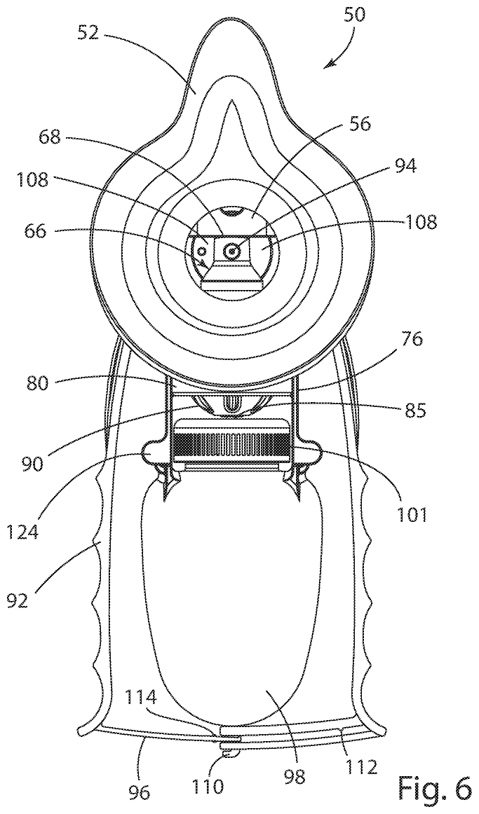

Compressed air chamber 98 may in some embodiments also be charged (or even recharged) using an access port 106 (See FIG. 5) such as an optional valve stem known in the arts for vehicle tires and the like. One embodiment the compressed air chamber 98 is configured to hold up to 0.1-120 PSI, preferably in the range of 20-60 PSI of air.

Accordingly, a bicycle pump or other available compressed air generator could be used to charge the chamber. Compressed air chamber 98 is also sized to allow for the desired amount pressure to force the desired amount of medication 104 through the jet housing. As shown in FIGS. 10, once air 46 is forced into the medication chamber 79, medication 104 is forced up aerosol channel 88 and into the nebulizer channel 66 where it can exit at nebulizer housing orifice 94 (a pressure release orifice). The pressure release orifice 94 is configured to produce aerosolized fluid of varying droplet/particle sizes ranging from 1-10 micrometers in diameter, preferably ranging from 3-5 micrometers in diameter. Pressure release orifice may have a diameter of 0.35-2.0 mm, for example.

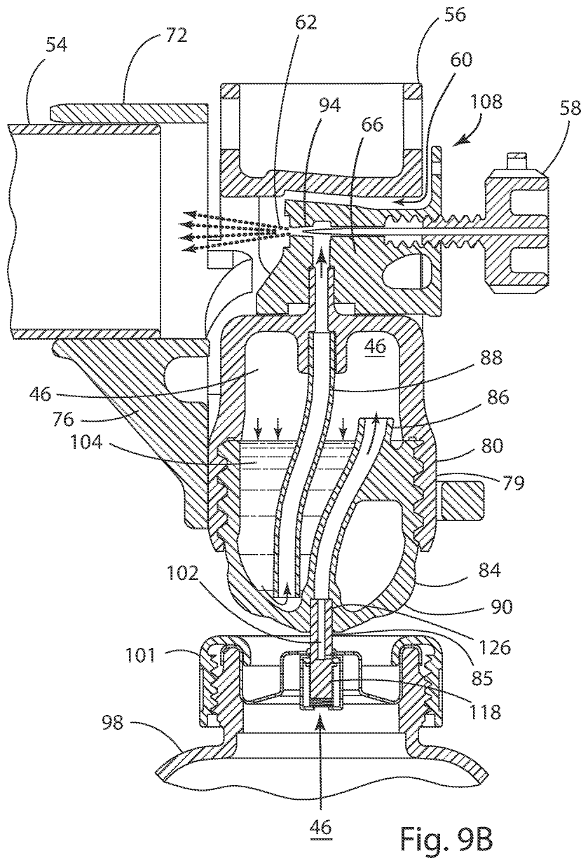

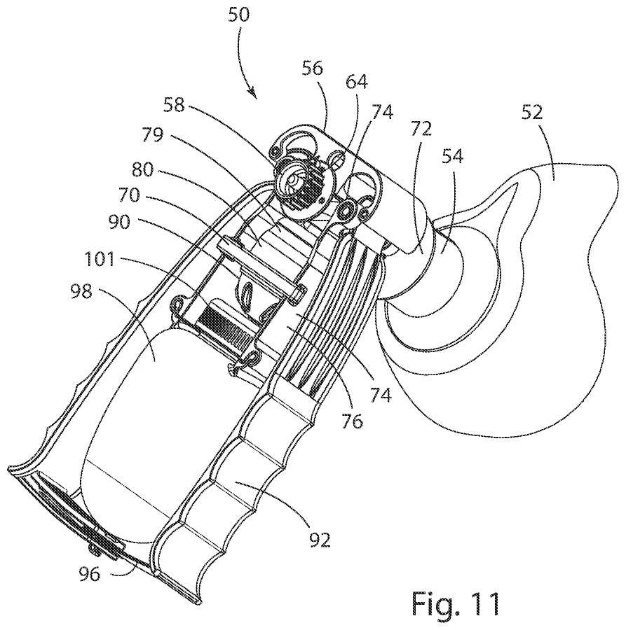

To provide nebulization, disposed within nebulizer housing orifice 94 is a retractable nebulizer jet needle 62. Droplet size control variation ‘knob’ 58 holds the nebulizer jet needle 62 and is threaded 60 into the nebulizer housing 66 causing the forward and backward motion of the nebulizer jet needle 62 in the housing (See, FIGS. 9A and 9B). As nebulizer needle 62 is retracted, the opening of orifice 94 becomes greater due to the taper of the needle. This results in smaller atomization droplets when needle 62 is threaded forward into housing 66 (FIG. 9A) and larger atomization droplets when thread 60 is turned backward out of housing 66 (FIG. 9B). Medication 104 exits as a mist and combines with ‘fresh’ air 108 drawn along the open sides around nebulizer housing 66. Droplet sizer Indicators 64 (FIG. 11) may be optionally added to guide a user of the droplet size control knob.

Presented herein are compositions and methods related to site-specific delivery of a pharmaceutically active compound to the respiratory tract of an animal/human, and in particular compositions and methods related to delivery of an approved pharmaceutically active compound (“drug”) with antiviral activity to a viral infection site of the respiratory tract which synergistically maximizes interaction of the drug with extracellular virus particles, inhibits/reduces viral epithelial cell entry through potential interaction with sites of extracellular viral binding to epithelial cell membranes, and potentiates drug partitioning into epithelial cells.

For clarity, the following terms are defined as they apply herein:
“Aerodynamic diameter” is the diameter of a particle with unit density that settles at the same velocity as the particle in question under the influence of gravity.
“Aerosol” means a suspension of particles in a gaseous medium, (e.g., air) and a solvent.
An “aqueous aerosol” is an aerosol formed from an aqueous solution (i.e., a solution containing water as a solvent).
“Chemical stability” refers to the stability of the drug compound itself. To be chemically stable, the chemical structure remains constant and doesn't degrade.
“Physical stability” refers to the drug staying in solution, as desired for the formulation. To be physically stable, the drug cannot denature or come out of solution or otherwise lose the integrity of the desired formulation.
“Functional stability” refers to the stability of the formulation when used in an aerosolization device. To have functional stability, good aerosol performance must be achieved consistently. The aerosol generated has the same attributes, e.g., consistent viable fraction throughout.
“Emitted dose” or “ED” is the amount of aerosolized particles of the active ingredient (e.g., recombinant human interferon alpha-2b) that is emitted from a drug delivery device. “Mean emitted dose” is an arithmetic average of the emitted doses released over a repetition of a plurality of deliveries under the same conditions.
“Fine particle fraction” or “FPF” is the fraction of particles in an emitted dose that are of a size capable of reaching the deep lung or alveolar membranes. Unless otherwise indicated, fine particle fraction is calculated herein as that fraction of the particles which are less than or equal to about 3.5 microns as measured by a Cascade Impactor, light scattering methods, phase Doppler particle sizing or other applicable methods.
“Fine particle dose” or “FPD” is the amount of the active ingredient that actually reaches the target zone (i.e., deep lung, alveolar membranes) and is a product of emitted dose and fine particle fraction (i.e., FPD=ED×FPF).
“Mass median aerodynamic diameter” or “MMAD” is the aerodynamic diameter of the particle where 50% of the aerosol mass is in larger particles and 50% of the aerosol mass is in smaller particles.
“Particle size distribution” or “PSD” is a description of the way the mass of the aerosol is distributed across the range of aerosol particle sizes.
“Dosage form” or “DF” is a container closure system that is used to hold a dose (or partial dose) of a formulation prior to aerosolizing it.
“Physical/chemical” or “P/C” identify properties of and changes in matter as physical or chemical.
“Pharmacokinetics” or “PK” refers to the movement of drugs through the body, i.e., the study and characterization of the time course of drug absorption, distribution, metabolism and excretion.
“Pharmacodynamics” or “PD” refers to the study and characterization of the biochemical and physiological response to drugs and their mechanism of action.
“Microbe free” refers to the formulation being rendered free from microorganisms by aseptically passing it through a sterilized microbial retentive filter membrane.
“System efficiency” is defined as the portion of the drug in the container-closure system that reaches systemic circulation.
“Bioavailability” refers to the portion of the emitted or delivered or inhaled dose from the container-closure system that reaches the intended site of pharmacological activity. Specifically, as used herein “bioavailability” is the rate and extent of the amount of administered drug that reaches the site of action (pharmacologic activity). For systemically administered drugs (IV, IP, IM, PO, etc.), the rate and extent of drug reaching the systemic circulation is used as a surrogate for the rate and extent of drug reaching the site of pharmacological activity because (1) the drug must enter the systemic circulation to reach the site of action, and (2) it is usually very difficult to measure drug concentration at the site of pharmacological activity compared to measuring blood or plasma drug concentrations. For locally administered drugs, such as herein, the drug DOES NOT need to reach the systemic circulation prior to reaching the site of pharmacological activity and blood/plasma drug concentrations are not representative of those at the site of action.
“CQ”, “HCQ”, and “ADQ” are 4-aminoquinolones somewhat related to the natural compound quinine.
“CQ” refers to chloroquine.
“HCQ” refers to hydroxychloroquine, an antimalarial with proven efficacy for treating inflammatory conditions including pulmonary disease states.
“ADQ” refers to amodiaquine.
“Liposomes” refer to microscopic vesicles, each consisting of an aqueous core enclosed in one or more phospholipid layers and used to convey vaccines, drugs, enzymes, or other substances to target cells or organs.
“Lysosomes” refer to organelles in the cytoplasm of eukaryotic cells which have degradative enzymes enclosed in a membrane.
“Sustained release” refers to the gradual release of an active agent over a period of time, allowing for a sustained effect or prolonged action.
“Partition coefficients” refers to the ratio of concentrations of a compound in a mixture of two immiscible solvents at equilibrium. Thus, it relates to a comparison of the solubilities of the solute in these two liquids or in other words the distribution of a given agent at equilibrium between two substances at the same temperature, pressure, and volume. A partition coefficient of a molecule between octanol and water is a standard measure of lipophilicity.
Log P refers to the logarithm of the octanol/water partition coefficient of a compound.
“Lipophilicity” refers to the affinity of a drug for a lipid environment.
“Site of pharmacological activity” refers to the initial interaction of a drug with cells at the site of action; the resultant physiological and biochemical consequences are the drug effects.
“Lung Airway Chips” or “LAC” refers to an alternative to animal testing and is a microfluidic tool to model complex and dynamic inflammatory responses of healthy and diseased lungs in vitro. The device is made using human lung and blood vessel cells and it can predict absorption of airborne nanoparticles and mimic the inflammatory response triggered by microbial pathogens. It can be used to test the effects of environmental toxins, absorption of aerosolized therapeutics, and the safety and efficacy of new drugs.
“Liquid complex” refers to a combination of a pharmaceutically active ingredient with an inactive excipient in a 1:2 to 2:1 mass ratio such that the combination is a homogenous, and preferably lipophilic liquid at body temperature.
“Eutectic” refers to or denotes a mixture of substances (in fixed ration) that melts and solidifies at a single temperature that is lower than the melting points of the separate constituents or of any other mixture of them.
“Excipient” refers to an inactive substance that serves as the vehicle or medium for a drug or other active substance.
“Surfactant” refers to a substance which tends to reduce the surface tension of a liquid in which it is dissolved.
“Micelle” refers to a macromolecular aggregation of surfactant molecules, typically below about 100 nm in geometric diameter, that are homogenously distributed in a vehicle. Herein, this specifically will refer to aggregates of surfactants in which the non-polar portions of the surfactant are associated in the interior of the aggregate creating a lipophilic environment with the polar portions of the surfactants exposed on the exterior of the aggregate to interact with the water vehicle.
“Micellar solution” refers to use of surfactant micelles to create a stable and homogenous and sub-micron aqueous dispersion of an otherwise water insoluble molecule or liquid complex through partitioning of the latter into the lipophilic interior of a surfactant micelle.
“Peroral” refers to ingestible dosages such as by “pill” (an ingestible dosage form produced by rolling a “dough” containing the drug into a ball and drying) tablets, capsules, “gummies”, elixirs, and suspensions.
   (See also generally, US Pub 2008/0138397 A1 to Schuster et al. and Wikipedia)

To assist in the understanding of the present embodiments, the structure and physical/chemical (P/C) and pharmacokinetic (PK) properties of chloroquine (CQ) and its two analogs, hydroxychloroquine (HCQ) and amodiaquine (ADQ), as well as in vitro data of these compounds in cellular models of SARS CoV-2 infection, efficacy data in animal models of SARS CoV-2 infection, and the results of human clinical trials in COVID-19 patients are provided herein. The in vivo evaluations have all involved systemic drug administration. An analysis of the potential impact of drug P/C properties and drug PK properties upon the variable results of the in vitro, animal, and clinical studies is also provided. Mechanisms of the ability of these drugs to inhibit SARS CoV-2 in vitro, which are proposed in the literature, are also provided along with alternate drug administration studies suggested in the literature and registered in the US government's Clinical Trials website to improve the clinical efficacy of these drugs in COVID-19 patients.

Drug Properties

CQ, HCQ, and ADQ are 4-aminoquinolones somewhat related to the natural compound quinine and are traditionally used to prevent and treat malaria. These drugs are also used to treat liver infection caused by protozoa (extraintestinal amebiasis). Side effects may include heart rhythm problems such as QT prolongation, ventricular fibrillation, ventricular tachycardia, (Mayo Clinic Website). HCQ is considered to produce about 40% of the total side effects associated with CQ. All three drugs were developed in the 1930's and 1940's and CQ and HCQ are currently approved as prescription drug products for peroral administration in the U.S. AMQ was withdrawn from use in the United States due to rare occurrence of agranulocytosis and liver damage with high doses or prolonged treatment (referenced in Si, 2020).

Typical malaria treatment doses are 1000 mg (CQ) or 800 mg (HCQ, ADQ) on day 1, followed by 500 mg (CQ) or 400 mg (HCQ, ADQ) at 6, 24, and 36 hr after the first dose. (Mayo Clinic Website) All three drugs are administered perorally. The structures of CQ, HCQ, and ADQ as well as the active metabolite of the latter, desethylamodiaquine (DEAQ), are shown in FIG. 1 and a summary of pertinent P/C and PK parameters is in Table 1.

The probable sites of the molecule associated with efficacy (the conjugated ring structure and tertiary amine structure at the ends of the molecule) are essentially the same—HCQ has a hydroxy group on one of the ethyl groups of tertiary amine group. As shown in Table 1, this substitution slightly increases the hydrophilicity of HCQ relative to CQ. More importantly, ADQ differs from CQ and HCQ by having an aromatic ring containing a hydroxyl group between the secondary and tertiary amine structures instead of an isopentyl aliphatic structure. This difference appears to be responsible for the greatly reduced lipophilicity (as indicated by the log P values) and increased water solubility of ADQ relative to CQ and HCQ.

TABLE 1
Drug physical/chemical and pharmacokinetic properties (IV in humans)

| | CQ | HCQ | ADQ | DEAQ |
|---|---|---|---|---|
| Physical/Chemical | | | | |
| Molecular Weight (g/mol) | 319.9 | 335.9 | 355.9 | 327.8 |
| Log P | 4.6 | 3.9 | 2.6 | 2.3 |
| Water Solubility (mg/mL) | 0.00014 | 0.0261 | 1-10 | N/A |
| Pharmacokinetic | | | | |
| T_{1/2} (hr) | 569 | 630 | 2 | N/A |
| V₂ (L) | 7,973 | 26,711 | 39 | N/A |
| References | | | | |
| P/C | PubChem | PubChem | PubChem | PubChem |
| PK | (Aderoumnu, 1986) | (Tett, 1988) | (White, 1987) | |

Although structures and properties of the free bases are shown above, CQ is primarily administered as the diphosphate salt and HCQ and ADQ as the dichloride salts. The free bases of CQ and HCQ are extremely lipophilic with log P values of 4.6 and 3.9 and exhibit very low water solubilities compared to the ADQ free base. Since the log P values of CQ and HCQ are well above the optimal values for peroral absorption of 1-3, it is likely that these compounds exhibit poor partitioning into and diffusion through tissue. Further, the very high volumes of distribution of CQ and HCQ indicates extensive binding and distribution to lipoidal tissues. Hence, these properties would tend to predict that ADQ will partition into and through multicellular tissues much more readily than CQ and HCQ.

Efficacy of CQ, HCQ, and ADQ in Preclinical Models of SARS-CoV-2 Infection and Human Clinical Studies.

CQ, HCQ, and ADQ have all exhibited good activity against SARS-CoV-2 infection in in vitro studies using either Huh-7 cells which are derived from a human liver tumor (Si, 2020) and Vero-6 cells derived kidney tissue of an African green monkey. (Osada, 2014). Huh-7 cells only express low levels of ACE2 and they do not express TMPRSS2, (Si, 2020) both of which are involved in SARS-CoV-2 cell infection. Verc-6 cells express both receptors. Table 2 summarizes the results of reported evaluations of CQ, HCQ, and AMQ in these in vitro cell models of SARS-Cov-2 infection.

The Huh-7 studies reported by Si, et al. employed SARS-CoV-2 pseudoparticulates (pp) instead of SARS-CoV-2 virus and therefore could only evaluate inhibition of viral entry into these cells. (Si, 2020) The studies employing Vero-6 used SARS-CoV-2 virus strains and evaluated other measures of infection, primarily virus titer in the infected cells. Various modes of drug presentation were used including pre-treatment with drug prior to and often subsequent to viral infection, treatment after viral infection, and treatment concurrent with viral infection.

Nonetheless, the results presented in Table 2 indicate that the three drugs and active metabolite ADQ all exhibit very good antiviral activity for SARS-CoV-2 across the several protocols employed in these cell culture models. One aspect of the latter that should be noted is that the bioavailability of the drugs at the site of pharmacological activity, considered to be the cellular surfaces and interior is high as it depends only upon the relative abilities of the individual drugs to adsorb to the cellular surface, interact with extracellular virus, and partition into the intracellular structures under conditions of static exposure, i.e., in the cell culture media.

TABLE 2
Summary of reported evaluations of CQ, HCQ, and ADQ in cell
culture models of SARS-CoV-2 (or similar) infection

| Reference | Drug | Cell Type | Infection | Treatment | Effect | Result |
|---|---|---|---|---|---|---|
| Si, | CQ | Huh-7 | SARS-CoV-2pp | Concurrent | Inhibition of | 50% entry @ 5 μM; |
| 2020 | | | | | viral entry | 10% entry @ 1 μM |
| | HCQ | Huh-7 | SARS-CoV-2pp | Concurrent | Inhibition of | 50% entry @ 5 μM; |
| | | | | | viral entry | 30% entry @ 1 μM |
| | ADQ | Huh-7 | SARS-CoV-2pp | Concurrent | Inhibition of | 30% entry @ 5 μM; |
| | | | | | viral entry | 10% entry @1 μM |
| | ADQ | Vero 6 | SARS-Cov-2 | Pretreatment | Inhibition of | IC₅₀ 10.3 μM |
| | | | | | infection | |
| | DEAQ | Vero 6 | SARS-Cov-2 | Pretreatment | Inhibition of | IC₅₀ 8.5 μM |
| | | | | | infection | |
| Yao, | CQ | Vero 6 | SARS-Cov-2 | Treatment | Viral | EC₅₀ 23.9 μM @ 24 hr |
| 2020 | | | | | Replication | EC₅₀ 5.47 μM @ 48 hr |
| | HCQ | Vero 6 | SARS-Cov-2 | Treatment | Viral | EC₅₀ 6.14 μM @ 24 hr |
| | | | | | Replication | EC₅₀ 0.72 μM @ 48 hr |
| | CQ | Vero 6 | SARS-Cov-2 | Pretreatment | Viral | EC₅₀ >100 μM @ 24 hr |
| | | | | | Replication | EC₅₀ 18.01 μM @ 48 hr |
| | HCQ | Vero 6 | SARS-Cov-2 | Pretreatment | Viral | EC₅₀ 6.25 μM @ 24 hr |
| | | | | | Replication | EC₅₀ 5.85 μM @ 48 hr |
| Liu, | CQ | Vero 6 | SARS-Cov-2 | Pre- and | Viral | EC₅₀ 7.36 μM @ 90 m |
| 2020 | | | | Post- | Replication | |
| | | | | treatment | | |
| | HCQ | Vero 6 | SARS-Cov-2 | Pre- and | Viral | EC₅₀ 12.96 μM @ 90 m |
| | | | | Post- | Replication | |
| | | | | treatment | | |
| Wang, | CQ | Vero 6 | SARS-Cov-2 | Concurrent | Viral | EC₅₀ 1.13 μM @ 48 hr |
| 2020 | | | | | Replication | |

In addition to reporting cell culture results, Si, et al. describe the development of a three-dimensional in vitro lung tissue mimic structure using microfluidic culture technology termed Lung Airway Chips (LAC). (Si, 2020) The LAC (FIG. 2) has a membrane for cell growth between air and fluid (for culture media) channels. This permits growth of lung tissue epithelia representative of both that interfacing with air in the lumen of the lung (mucociliary, pseudostratified epithelium with proportions of airway-specific cell types such as ciliated cells, mucus-producing goblet cells, club cells, and basal cells) and that interfacing with biological fluids (microvascular endothelium with a continuous planar cell monolayer with cells linked by VE-cadherin containing adherens junctions). The LAC were reported to mimic the pathology of several influenza viral strains when these were introduced into the airway channel and the pharmacological activity of approved influenza antiviral drugs when these were introduced into the fluid channel in a manner to mimic plasma concentrations following systemic (peroral) administration of the drugs. Therefore, this model approximates any bioavailability effects associated with peroral and other systemic routes of administration dosing of test drugs.

Si, et al. used the LAC model to evaluate the ability of pharmaceutical salts of CQ (phosphate), HCQ (sulfate), and ADQ (hydrochloride) to inhibit the entry of SARS-CoV-2 pseudoparticulates (administered via airway channel). The evaluation protocol consisted of perfusion of the fluid channel of the LAC with drug at human blood maximum concentrations (clinically used doses) for 24 hour at a assumed flow rate of 60 μL/hr (actual flow rate not reported, but this is the flow rate employed in previous studies using the LAC) along with introduction of 30 μL of the drug at the same concentration statically (volume not reported, but this the volume used for subsequent introduction of drug and SARS-CoV-2pp). After 24 hr, SARS-CoV-2pp was introduced into the LAC airway channel in 30 μL medium containing the drug (same concentrations as in fluid channel and previous treatment of airway channel) with static incubation for 48 hour concurrent with continued perfusion of drug through the fluid channel. Hence, drug was available at the air interface of the epithelial cells along with the SARS-CoV-2pp as well as at the surfaces on the porous membrane in contact with the drug containing media in the fluid channel. Despite this “double treatment”, only ADQ hydrochloride inhibited SARS-CoV-2pp entry into LAC epithelial cells as measured by qPCR quantitation of viral mRNA in the epithelial cells. The authors then demonstrated that ADQ hydrochloride was able to reduce SARS-CoV-2 viral load in a COVID-19 hamster model as measured by RT-qPCR of the viral N transcript after Intraperitoneal (IP) administration (systemic) whereas HCQ sulfate was ineffective thereby indicating that the LAC model was predictive of in vivo performance of aminoquinoline drugs although drug administration in the LAC model involved presentation at both epithelial cell interfaces which is not representative of systemic delivery. However, it should be noted that the LAC model was limited to evaluation of drug effects upon viral cell entry whereas the in vivo hamster model would have included this antiviral mechanism of activity as well as any other putative antiviral mechanisms of activity.

One probable explanation for the differing effects of CQ/HCQ and ADQ in the cell culture efficacy models and the LAC lies in the physical/chemical properties of these compounds, specifically pertaining to the Log P values and water solubilities of the free base species of these drugs, and the impact of the latter upon drug bioavailability at the surface of and within the epithelial cells in the LAC. The pH partition theory predicts that only the non-ionized free base of each of these drugs will partition into the epithelial cells. The free bases of CQ and HCQ are extremely lipophilic with log P values of 4.6 and 3.9 and exhibit very low water solubilities compared to the free base of ADQ. Since the log P values of the free bases of CQ and HCQ are well above the optimal values for peroral absorption of 1-3 and have very low water solubilities, it is likely that these compounds will exhibit poor partitioning into and diffusion through tissue. Hence, these properties would tend to predict that ADQ with a free base Log P of 2.6 and a relatively high-water solubility will partition into cells and diffuse through multicellular tissues much more readily than CQ and HCQ. Since all three of the above aminoquinolines exhibit similar in vitro cell culture antiviral activity, the lack of efficacy of CQ phosphate and HCQ sulfate in the three-dimensional LAC in vitro model and animal models is almost certainly due to low bioavailability of CQ and HCQ administered as pharmaceutical salts at the site of viral infection relative to AQ similarly administered.

No reports of preclinical animal studies of the effects of CQ against SARS-CoV-2 infection are presently known. One study of ADQ hydrochloride in hamsters, three studies of HCQ sulfate in hamsters, and one study of HCQ sulfate in Rhesus Macaque monkeys have been reported and are summarized in Table 3. In the one report involving ADQ hydrochloride evaluation it was found to be effective as a prophylactic treatment of 50 mg/kg administered subcutaneously (SC) QD in reducing SARS-CoV-2 infection from virus introduced either intranasally to hamsters or by transfection of healthy animals through association with nasally infected animals. However, HCQ sulfate was ineffective in similar prophylactic treatment protocols in both nasally infected hamsters and in hamsters infected by transfection at an IP dose of 50 mg/kg QD (600 mg/kg loading dose in the transfected protocol) in a study reported by Kapstein, et al. (Kapstein, 2020). HCQ sulfate was also ineffective in a study using a similar prophylactic protocol with HCQ IP doses of 50 mg/kg QD reported by Rosenke, et al. and also in a therapeutic protocol using IP doses of 50 mg/kg QD. Finally, HCQ sulfate was ineffective in both prophylactic and therapeutic protocols in Rhesus Macaque monkeys using peroral (PO) doses of 6 mg/kg QD.

TABLE 3
Summary of reported preclinical animal studies involving
ADQ and HCQ in models of SARS-CoV-2 infection

| Reference | Drug | Animal | Infection | Protocol | Result |
|---|---|---|---|---|---|
| Si, | ADQ | Hamster | Nasal on day 2 | 50 mg/kg SC day (1 day | 70% reduction in |
| 2020 | | | | before infection) | viral load on day 4 |
| | | | | and days 2, 3, 4 | |
| | | | Exposure of | 50 mg/kg SC day 1 (1 day | 90% reduction in |
| | | | healthy | before exposure to infected | viral load on day 4 |
| | | | animals to | animals) and days 2, 3, 4 | |
| | | | nasally | | |
| | | | infected | | |
| | | | animals on | | |
| | | | Day 2 | | |
| Kapstein, | HCQ | Hamster | Nasal on day 2 | 50 mg/kg IP day (1 day | No reduction in |
| 2020 | | | | before infection) | viral load on day 4 |
| | | | | and days 2, 3, 4 | |
| | | | Exposure of | 600 mg/kg IP day 1 (1 day | No reduction in |
| | | | healthy | before exposure to infected | viral load on day 5 |
| | | | animals to | animals infection) and 50 | |
| | | | nasally | mg/kg IP days 2, 3, 4, 5 | |
| | | | infected | | |
| | | | animals on | | |
| | | | Day 2 | | |
| Rosenke, | HCQ | Hamster | Nasal on day 2 | 50 mg/kg IP day (1 day | No reduction in |
| 2020 | | | | before infection) | viral load or change |
| | | | | and days 2, 3, 4 | in disease |
| | | | | | progression |
| | | | Nasal on day 1 | 50 mg/kg IP day (1 hr after | No reduction in |
| | | | | infection) and days 2, 3 | viral load or change |
| | | | | | in disease |
| | | | | | progression |
| | | Rhesus | Combination | 6 mg/kg PO on | No effect upon viral |
| | | Macaque | of four routes | days −9, −2, and 5 | loads at days 3, 5, |
| | | Monkey | (intratracheal, | | or 7, or disease |
| | | | oral, | | pathology through |
| | | | intranasal and | | post-mortem on |
| | | | ocular) on day | | day 7. |
| | | | 0. | | |
| | | | Combination | 6 mg/kg PO 12 18, 36, 60, 84 | No effect upon viral |
| | | | of four routes | and 108 hours post-infection | loads at days 3, 5, |
| | | | (intratracheal, | | or 7, or disease |
| | | | oral, | | pathology through |
| | | | intranasal and | | post-mortem on |
| | | | ocular) on day | | day 7. |
| | | | 0. | | |

Rosenke, et al. have also summarized clinical experience with HCQ for treatment of COVID-19, the disease associated with SARS-CoV-2 infection as follows: “HCQ has been promoted as a COVID-19 treatment option and became part of multiple large-scale clinical trials including one of four initial treatment options in the multinational WHO “Solidarity” clinical trial for COVID-19 (WHO, 2020). However, HCQ treatment does not come without risks as the 4-aminoquinolines are associated with multiple adverse effects such cutaneous adverse reactions, hepatic failure, and ventricular arrythmia; overdose is also difficult to treat (AHFS, 2020). The US FDA recently updated its guidance by waming against use of HCQ outside of the hospital setting because of the potential for serious adverse effects (US FDA, 2020). Further, “the WHO Solidarity study . . . ” has “been excluded HCQ arms due to a lack of evidence for therapeutic efficacy, and an increase level of adverse effects in COVID-19 patients (WHO, 2020).”

Analysis of Reported Efficacy Results of CQ, HCQ, and ADQ Preclinical Models of SARS-CoV-2 Infection and Human Clinical Studies

Therefore, despite a number of studies documenting the effectiveness of HCQ against SARS-CoV-2 infection in in vitro cell culture models, the drug has repeatedly failed in (1) an in vitro three-dimensional tissue model requiring partitioning of HCQ into epithelial cells to reach the presumed site(s) of pharmacological activity and in (2) preclinical animal models using systemic dosing (IP and PO) which requires circulatory biodistribution and tissue diffusion to reach the presumed site(s) of pharmacological activity in the lung. However, although the efficacy of ADQ in in vitro cell models of SARS-CoV-2 infection is similar to that of HCQ, this drug DID exhibit prophylactic efficacy against SARS-CoV-2 infection in hamsters at the same systemic dose of 50 mg/kg SC QD as used for HCQ sulfate dosing in hamster studies. This comparison clearly indicates that ADQ is more bioavailable at the putative sites of action in the in vitro three-dimensional tissue model and in vivo in animal models.

Kapstein, et al. also measured HCQ lung concentrations post-mortem in their hamster study and found that while the endosomal/lysosomal concentrations were quite high, the cytosolic and interstitial HCQ concentrations were well below the estimated EC50. The authors point out that:
“Although alkalization of endosomes has been proposed as one of the key mechanisms of the broad-spectrum antiviral effect of HCQ, the mechanism of action against SARS-CoV-2 has not been completely unraveled (See discussion below). Therefore, the very low cytosolic concentrations of HCQ in the lung may explain the absence of an antiviral effect of HCQ against SARS-CoV-2 in vivo.”

CQ and HCQ have similar P/C, PK, and antiviral activity against SARS-CoV-2 in in vitro cell culture models and it would be expected that CQ would also be ineffective (as is HCQ) when administered systemically (e.g., through injection or peroral) in animal models, particularly given its lack of efficacy in an in vitro three-dimensional tissue model (LAC). Since another 4-aminoquinoline drug, ADQ, with differing P/C properties more favorable to drug tissue partitioning and diffusion and PK properties suggestive of less extensive binding to lipoidal tissue (adipocytes), but with similar in vitro SARS-CoV-2 antiviral activity does exhibit efficacy in an animal model, a reasonable conclusion is that CQ (and HCQ) has insufficient bioavailability at the putative site(s) at which it can affect SARS-CoV-2 infection and/or replication when administered systemically.

Proposed Mechanisms of Aminoquinoline Drugs SARS CoV-2 Antiviral Activity

CQ has been shown to exhibit in vitro antiviral activity against RNA viruses as diverse as rabies virus, poliovirus, HIV, hepatitis A virus, hepatitis C virus, influenza A and B viruses, influenza A H5N1 virus, Chikungunya virus, Dengue virus, Zika virus, Lassa virus, Hendra and Nipah viruses, Crimean-Congo hemorrhagic fever virus and Ebola virus, as well as various DNA viruses such as hepatitis B virus and herpes simplex virus. (reviewed in Devaux, 2020). Based upon the mechanisms of action of CQ against the above viruses, Devaux, et al. reviewed the modes of action of CQ that may be responsible for its demonstrated in vitro antiviral activity against SARS-CoV-2 in cell culture models. These may be summarized as follows: (Devaux, 2020)
Inhibition of a pre-entry step of the viral cycle by interfering with viral particles binding to their cellular cell surface receptor.
Impairment of an early stage of virus replication by interfering with the pH-dependent endosome-mediated viral entry of enveloped viruses. The mechanism of inhibition likely involves the prevention of endocytosis and/or rapid elevation of the endosomal pH and abrogation of virus-endosome fusion. A pH-dependent mechanism of entry of coronavirus into target cells has been reported for SARS-CoV-1 after binding of the DC-SIGN receptor, which may translate to SARS-CoV-2 entry into lung cells after binding to ACE and TMPRSS2 receptors.
Chloroquine can also interfere with the post-translational modification of viral proteins, which involve proteases and glycosyltransferases and occur within the endoplasmic reticulum or the trans-Golgi network vesicles, may require a low pH.

These putative mechanisms of action of CQ against SARS-CoV-2 suggest that drug should be present in sufficiently effective amounts at the epithelial membranes of alveoli lung tissue to putatively affect SARS-CoV-2 binding to epithelial ACE and TMPRSS receptors and pH mediated epithelial membrane fusion for entry into the lung cells, and also at both epithelial and epithelial membranes to facilitate partitioning of the drug into the lung cells to exert effects intracellularly.

Present Embodiments for Aminoquinoline COVID-19 Treatment Strategy by Pulmonary Aminoquinoline Administration

Since CQ (or HCQ) is apparently not sufficiently bioavailable at the epithelial membranes of alveoli cells after systemic administration of their pharmaceutical salts (e.g., through injection, ingestion of peroral dosage forms, and the like) due to its P/C and PK properties, the present embodiments provide methods and compositions for pulmonary administration of CQ (or HCQ) free base. Pulmonary delivery of CQ (or HCQ) free base provides direct contact of administered drug free base with the alveoli epithelial membranes to permit activity against viral receptor binding and potential pH mediated viral—alveoli epithelial membrane fusion to reduce viral entry into alveoli cells. Drug free base concentrations in contact with the alveoli epithelial membrane resulting from successful pulmonary delivery are higher than those reaching the epithelial membrane from systemic administration and provide the potential for increased intracellular partitioning of the drug.

There are over 65 different approved drug products (twenty plus active ingredients) utilizing pulmonary delivery, (Labris, 2003) including many directly targeting lower respiratory infections. (Zhou, 2015) The latter include both nebulized liquid systems and dry powder systems with defined particle size distributions. Some articles have even generally proposed the use of pulmonary delivery with CQ and HCQ, albeit using the pharmaceutical salts thereof that are primarily used for peroral administration. (Fassihhi, 2020; Kimke, 2020; Kavanagh, 2020) The referenced articles describe potential use of nebulized aqueous solutions of HCQ disulfate or other water-soluble salt.

Reported human pulmonary administration HCQ sulfate include Phase I and Phase II trials to assay safety and efficacy in treatment of asthma (Kavanah, 2020) and a personal use study (Kimke, 2020).

Kimke, et al. described a personal use study as follows:
“Since there is currently no commercial aerosol application available, two of the authors (A.K., clinical director and B.W., medical student working at an ICU with acute COVID-19 patients) decided to test tolerability and possible side effects of the inhalation of HCQ, starting with a dosage of 1 mg b.i.d. (dissolved in 2 ml of 0.9% NaCl solution and using a commercial nebulizer generating a droplet size <5 μm which can reach the alveolar space) which was stepwise increased up to 4 mg per day over a period of one week.

Inhalation was well tolerated without relevant side effects. The only observation was after 4 days the feeling of a transient bitter taste in the mouth which lasted 2-3 h after the inhalations.”

Kavanah, et al. include information from Phase I and Phase II clinical trials of nebulized hypertonic 100 mg/mL HCQ sulfate to assay safety and efficacy in treatment of asthma conducted for Adradigm Corporation in 2004 and 2006. Although the Phase II study did not meet its clinical endpoints for asthma, the two studies demonstrated that pulmonary doses of up to 20 mg HCQ sulfate QD are well tolerated with minimal side effects. (Kavanah, 2020)

The authors also note that HCQ sulfate (as well as CQ and other quinoline derivatives) has an extremely and unpleasant bitter taste and that Adradigm applied for a US patent (US20080138397A1, abandoned).

The authors of Fasshihi SC, Nabar NR, Fassihi R, Novel approach for low-dose pulmonary delivery of hydroxychloroquine in COVID-19, Br J Pharmacol 177:4997 (2020) propose dosing an aqueous solution of about 10-20 mg hydroxychloroquine sulfate (7.7-15.7 free base equivalent) QD to the lungs via nebulization of a volume of about 3-5 mL which translates to a range of about 0.4 to 5.2 mg/mL free base. This differs significantly from the present embodiments of, for example, 3-4 doses of 3 mL of a 1 mg/mL micellar solution of chloroquine (could be hydroxychloroquine) base (not salt) formulated to create a liquid lipoidal system that is subsequent solubilized into a micellar surfactant solution. Care is taken to not create conditions under which a salt of the active pharmaceutical compound is formed. It is also probable that 6 mL is an insufficient volume to cover the surface area of the lungs at a 2 um average particle size.

Second, hydroxychloroquine has three basic functional groups with pKa values of <4.0, 8.3 and 9.7, two of which would be protonated at pH 7.4. Hence, that hydroxychloroquine is neutral at physiological pH is therefore subject to challenge. Furthermore, as a neutral species in an aqueous physiological system, it may be able to freely diffuse in cells. However, this neglects to account for the extremely low water solubility of the neutral free base which is 0.026 mg/mL. If most of the drug proposed by the Fasshihi et al. dosing system would be altered from the protonated sulfate salt to the free base upon administration into the lung, the drug would precipitate and not be available for absorption.

The described technology herein involves use of a liposomal formulation of HCQ or a pharmaceutical salt thereof interchangeably and therefore does not teach the advantages of administration of the HCQ free base to improve drug bioavailability on the surface of and within the alveoli cells.

With regard to liposomal HCQ administration to lungs, Tai, et al. report a PK study conducted in Sprague-Dawley rats using intravenous (IV) and intratracheal (IT) instillation of HCQ sulfate with IT of a liposomal formulation of HCQ sulfate. (Tai, 2020) As shown in FIG. 1 in the publication, the drug administered as a solution by IV or IT administration exhibited similar drug PK profiles in heart and blood, with IT administration producing higher lung drug concentrations over the first four hours. IT administration of the liposomal formulation produced consistently higher lung drug concentrations over the 72 hour of the study, and lower blood and heart drug concentrations over about 36 hr. Although IT administration differs from pulmonary administration, especially in having a much larger ratio of dosage volume to lung tissue surface area which could significantly affect systemic absorption of HCQ, the data in FIG. 3 suggest that (1) IT administration provides a much higher ratio of HCQ lung/blood concentrations, hence reduced system exposure; and (2) the liposomal formulation does provide sustained release of HCQ.

A recent study on inhaled hydroxychloroquine showed doses of up to 4 mg/day of the drug was well-tolerated (Br J Pharmacol. 2020; 177:4997-4998., Letter to the Editor) (https://bpspubs.onlinelibrary.wiley.com/doi/pdf/10.1111/bph.15167). Two other current clinical studies involving pulmonary administered HCQ are described in the ClinicalTrials.gov website as follows:
1. A Study to Evaluate the Safety, Tolerability and Pharmacokinetics of Orally Inhaled Aerosolized Hydroxychloroquine Sulfate in Healthy Adult Volunteers
Sponsor: Pulmoquine Therapeutics, Inc
Collaborator: Rockefeller University
Study Design:
This is a randomized, double-blind placebo-controlled Phase 1 single-dose dose-escalation study to assess the safety, tolerability and PK of oral inhalation of AHCQ (hydroxychloroquine sulfate based upon the indicated aqueous drug concentrations of 20 and 50 mg/mL) in healthy participants.
Escalating single doses of AHCQ will be studied in healthy participants. The study drug will be administered by inhalation through the mouth, and participants will be encouraged to exhale through the nose. The study drug, AHCQ, will be administered starting at an initial dose of 20 mg (Cohort A1, 1 mL of 20 mg/mL AHCQ solution) with proposed subsequent doses of 50 mg (Cohort A2, 1 mL of 50 mg/mL AHCQ).
Two dose levels are planned to be evaluated. Each cohort will comprise 8 participants (6 active, 2 placebo). Therefore, 16 participants will initially be planned to be enrolled in the study.
Additional participants may be enrolled if one or more enrolled participants do not complete the study.
Start Date: Jun. 25, 2020
Completion Date: Aug. 17, 2020
2. Development and Validation of “Ready-to-Use” Inhalable Forms of Hydroxychloroquine for Treatment of COVID-19
Sponsor: Mansoura University
Study Design:
The first 20 consecutive patients (group A) will be treated by oral antibiotics, supportive treatment and inhalable hydroxychloroquine (HCQ). (In day 1, an inhaled hydroxychloroquine sulfate dose of 12 mg will be taken via nebulization three times/day (TID) as a loading dose. Then, starting from day 2, same dose of 12 mg of inhaled hydroxychloroquine will be taken twice/day (BID) as maintenance dose for 5 days. The subsequent 20 consecutive patients (group B) will receive the same treatment of group A but without inhalable hydroxychloroquine (HCQ). At day 7, all patients of both groups will be evaluated by clinical, laboratory and chest CT parameters. Patients of group B who still show no clinical, laboratory or radiological improvement will continue treatment by adding inhalable hydroxychloroquine (HCQ). for another 7 days and re-evaluated at day 14. Patients of group A who still show no significant improvement at day 7 only will be re-checked at day 14.
Start Date: Jul. 15, 2020
Est. Completion Date: Aug. 15, 2020

The present embodiments use formulation strategies to rapidly potentiate the topical respiratory tract epithelial bioavailability of approved drugs with in-vitro viral activity, while showing no apparent in-vivo antiviral activity after systemic administration, by mitigating drug physical properties that adversely affect topical drug topical respiratory tract epithelial bioavailability. Advantages over the prior art include administration of non-ionized form of the drug, changing the physical form thereof from a solid to a lipophilic liquid with a high chemical activity of the drug (>0.5), and incorporation of said lipophilic liquid into a submicron micellar solution. These combine to increase the bioavailability of the drug on the surface of and within the alveoli cells. Also, a novel therapeutic strategy for the use of CQ in the prophylactic and therapeutic treatment of COVID-19 is provided.

Despite the evidence that chloroquine and hydroxychloroquine can inhibit the membrane fusion associated with coronaviruses cell entry, and subsequent respiratory infections, they may have side effects, including problems with vision, muscle damage, seizures, and low blood cell levels, when administered systemically (through injection or peroral). These side, or unwanted, effects are the result of systemic treatment using these compounds resulting in whole body exposure and an inability to control the duration of exposure. Thus, there is a desire and need for a site-specific delivery of a pharmaceutically active compound to the respiratory tract of an animal/human.

To address these issues, the present embodiments use chloroquine and hydroxychloroquine compounds as the pharmaceutically active ingredients for delivery to the lower respiratory tracts and the lung periphery to be combined with an excipient and surfactant, which are chosen to provide effective antiviral activity in the targeted delivery region and for realizing the desired duration of release of the active ingredients. Optimization of this medication occurs by regulating, jointly through the choice of excipient and surfactant, the quality (absorption of drug with minimal levels of the excipient and surfactant to minimize absorption of these components) and quantity to achieve optimal effectiveness.

The present embodiments localize delivery of the formulations to targeted respiratory infections, for example, corona virus cell entry points in the lower respiratory tracts and the lung periphery. The present embodiments can deliver medication that target the site of effectiveness and optimally only the site of effectiveness. The formulations and delivery ensure the presence of the antiviral's active ingredients in optimized quality and minimal quantity and for an adequate duration in those regions (e.g., 1 day to 80 days assuming a 40 day half-life of tissue residence time) and ensures that the active ingredients be dispelled from the body after the targeted region of the respiratory tract has been treated sufficiently (e.g., in the case of chloroquine and hydroxychloroquine compounds to avoid the over-alkalinization of the lungs).

To illustrate one approach of the present embodiments, exemplary treatments of chloroquine and hydroxychloroquine compounds are demonstrated. To understand the mechanisms of action addressed by the present embodiments, we begin with lysosomes.

Lysosomes are organelles in the cytoplasm of eukaryotic cells which have degradative enzymes enclosed in a membrane. Lysosomes act as the waste disposal system of the cell by digesting in-use materials in the cytoplasm, from both inside and outside the cell. Material from outside the cell is taken-up through endocytosis, while material from the inside of the cell is digested through autophagy.

Cell entry of coronaviruses involves two principal steps: receptor binding and membrane fusion, the latter of which requires activation by host proteases, particularly lysosomal proteases. An important mode of action of chloroquine and hydroxychloroquine is the interference of lysosomal activity and autophagy. It is widely accepted that chloroquine and hydroxychloroquine accumulate in lysosomes (lysosomotropism) and inhibit their function and, to that degree, may inhibit the membrane fusion critical to the cell entry of coronaviruses. The effect of this is inhibiting the subsequent spread of respiratory infection, which is the primary cause of severe respiratory illness, morbidity and mortality through inhibited absorption of oxygen and release of carbon dioxide in the lower lungs, which include the trachea, the bronchi and bronchioles, and the alveoli. Diminished oxygen absorption leads to reduced muscle strength, including the muscles that enable the lower tract to draw inhaled air into the lower respiratory tract.

In one approach of the present embodiments, pulmonary site-specific pharmaceutical therapy uses drugs with antiviral activity formulated in aerosolized systems, such as aerosolized aqueous systems. The formulated solution uses a chloroquine compound and delivery mechanism together to meet these pulmonary delivery requirements mentioned herein.

Byway of example, in one embodiment aerosolized droplets of less than 5 μm are configured to penetrate reach deep lung sites. Nano-sized lipoidal particles containing approved drugs (less than 100 nm) may be stabilized with polymeric surfactants. This approach may be used, for example, to facilitate availability of lipoidal structures to interact with lipid coated SARS-CoV-2.

The dosage form of the present embodiments is formulated so that improvement of the bioavailability of drugs with SARS-CoV-2 antiviral activity in in-vitro cell-based assays at the site of infection will significantly enhance clinical efficacy. This requires pulmonary administration of a dosage form formulated to improve the absorption of such drugs into alveoli epical membranes. An ionizable, cationic drug such as chloroquine is typically administered perorally (approved dosage route) and pulmonarily (subject of experimental studies) as a salt (diphosphate in the case of chloroquine). The positive charge of the ionized species precludes apical absorption, and the low water solubility and very high lipophilicity of chloroquine base reduces both the amount and absorption of this species which is in equilibrium with the charged species in aqueous solution.

The primary formulation uses non-charged chloroquine base formulated in “liquid complexes” (typically 1:1 ratios with an “excipient” such as propylene glycol, USP) that are lipophilic and liquid at body temperature presented in a isotonic “micellar solution” using a surfactant suitable for pulmonary administration such as tocopheryl polyethylene glycol succinate (TPGS) (typically at a 4:1 mass ratio with the liquid complex). The amount of surfactant (TPGS for example) can be about 4:1 to the complex or about 8:1 to the drug. By way of a non-limiting example, one formulation may be 4 parts surfactant; 0.5 parts drug; and 0.5 excipient.

The ability to meet requirements for drug delivery to the lower respiratory tracts and the lung periphery to inhibit membrane fusion and for excipient and surfactant to be effective in the targeted region and for realizing the desired duration of release of the active ingredients depend in part on the partition coefficient of a molecule, a standard measure of lipophilicity. The mass flux of a molecule at the interface of two immiscible solvents is governed by its lipophilicity. The more lipophilic a molecule is, the more soluble it is in lipophilic organic phase at ambient temperature. For the same reason, drug penetration into a biological membrane is also influenced by the lipophilicity of the drug. However, since biological membranes consist of bilayer structures consisting of both lipid and water, there is a recognized range of partition coefficient values that favors drug penetration into these membranes. Partition coefficient values outside of this range indicate a molecule is either too hydrophilic (low partition coefficient values) or too lipophilic, i.e., insufficiently water soluble (high partition coefficient values) for efficient partitioning into biological membranes. To address localized delivery to targeted respiratory infection, to ensure the presence of the antiviral's active ingredients in sufficient quality and quantity and for an adequate duration in those regions, and to ensure that the active ingredients be dispelled from the body after the targeted region of the respiratory tract has been treated sufficiently, the present embodiments may aerosolize the formulation with a nebulizer with adjustable aerosolization droplet size in the range of 1-10 μm. The size of the aerosolized droplets have been shown to determine the depth to which the aerosolized medication solution will penetrate the respiratory system, with for example 5-10 μm, preferably 5-7 micron size droplets penetrating to the lower respiratory tract, and 1-4 micron size droplets penetrating to the lung periphery portion, including the ‘air sacs’ or alveoli, where the lungs and the blood exchange oxygen and carbon dioxide.

The Figure provides a simplified schematic of the human respiratory anatomy 208. As shown, the anatomy 208 provides a nose 210, a mouth 212, a lower respiratory portion 214, a lung periphery portion 216, a nasal cavity 218, an oral cavity 219, a pharynx 220, a larynx 222, a trachea 224, lungs 226 and bronchi 228. According to A simplified view of the effect of aerosol particle size on the site of preferential deposition in the airways (presented by Gardenhire, D.S. Rau's Respiratory Care Pharmacology. St. Louis: Elsevier, 2016), as aerosol particles are inhaled orally or through the nose: the larger particles/droplets (>10 μm) are filtered in the nasal cavity 18; >15 μm are filtered by the oral cavity 19; 5-10 μm generally reach the proximal generations of the lower respiratory portion 14, and particles/droplets of 1-5 μm reach to the lung periphery 16. Although particle/droplet size plays an important role in lung deposition, particle/droplet velocity and settling time are also a factor. Thus, the particle/droplet sizes of 1-5 μm of the presented compositions are preferred for reaching the lung periphery, and 5-10 μm particles/droplets are preferred for deposition in the conducting airways. Particles/droplets >10 μm (such as 10-100 μm particles/droplets) are preferred for deposition mostly in the nose. It is noted that the particle/droplet size, produced during aerosolization, determines the deposition site in the respiratory system. Though a particular size may be desired, in practice the aerosol contains a range of sizes following a normal bell-shaped distribution having particle/droplet sizes varying from 0 to 15 micrometers. Droplet size, and thus expected deposition site in the respiratory system, is impacted by pressure, spray pattern type, spray angle, nozzle type, fluid specific gravity, fluid viscosity and surface tension.

Also, to address localized delivery to targeted respiratory infection, to ensure the presence of the antiviral's active ingredients in sufficient quality and quantity and for an adequate duration in those regions, and to ensure that the active ingredients be dispelled from the body after the targeted region of the respiratory tract has been treated sufficiently, the present embodiments may aerosolize the formulation with respect to various viscosities. According to one approach, the viscosity of the formulation may lie about within a range of propyl alcohol needed to enable aerosolization to the targeted droplet size. Continuously variable droplet size, or particle size, within the range of 1 and 10 μm enables the delivery of the formulation in an aerosol to any specified depth of the respiratory system and allows targeting of that delivery to the region(s) of infectious activity. As shown in the following table, fresh water has a dynamic viscosity roughly half that of propyl alcohol; this formulation should have the viscosity of propyl alcohol +/−0.10 cP. It is noted that the aerosolization is impacted by fluid viscosity, propellant velocity and the surface geometry of the aerosolization device. Thus, this viscosity requirement may vary with both of the latter

| | Absolute Viscosity | | |
|---|---|---|---|
| Fluid | (N s/m², Pa s) | (centipoise, cP) | (10⁻⁴ lb/s ft) |
| Alcohol, ethyl (ethanol) | 0.001095 | 1.095 | 7.36 |
| Alcohol, methyl (methanol) | 0.00056 | 0.56 | 3.76 |
| Alcohol, propyl | 0.00192 | 1.92 | 12.9 |
| Water, Fresh | 0.00089 | 0.89 | 6.0 |

The use of pulmonary administration of aminoquinolines as cationic salts should provide significantly increased exposure of the lung epithelial membrane to the drug relative to systemic administration. However, although both HQ and HCQ have good peroral bioavailability (good absorption from the gastrointestinal (GI) tract with low first-pass hepatic metabolism) when administered as cationic salts, this may not be applicable to the lung. The GI tract has a relatively large luminal fluid volume, prolonged residence time, and extremely large surface area (due to the presence of villi and microvilli in the small intestine) that greatly facilitate absorption of compounds that are ionized in the luminal fluid. The relatively large volume of the latter maintains the drug species (ionized and small amount of un-ionized) in solution and available for interaction with the epithelial cells. Only the (very small) amounts of un-ionized drug molecular species that are in equilibrium with the ionized molecular species in solution partition into the enterocytes of the small intestine. Upon depletion of the un-ionized species by absorption, the equilibrium is re-established producing more un-ionized drug for further absorption. This process can occur throughout the prolonged GI tract residence time and can result in substantial absorption of drugs that are ionized in the GI luminal fluid due to the length of the residence time, the relatively large luminal fluid volume, and especially the extremely large absorbing surface area.

For the above reasons, application of cationic salt drugs as solutions to other sites of administration such as the lung, skin, oral cavity, and rectum typically results in poor absorption due to one or more of the above factors. Typical pharmaceutical practice is to administer the drug as an uncharged species dissolved in a suitable vehicle; the free base in the case of CQ and HCQ. Therefore, sufficient vehicle is needed to solubilize the target drug dose, and ideally, the chemical activity of the drug in the selected vehicle is high in order to provide the highest possible chemical potential for absorption. Unfortunately, drug saturation solubility in many pharmaceutically acceptable vehicles is typically not very high (rarely exceeding 10%), requiring high ratios of vehicle to drug. This would be disadvantageous in the lung where it is desirable to limit the amounts of exogenous material administered.

In addition to the factors mentioned above, CQ and HCQ free base have P/C properties, especially their high partition coefficient (Log P values) and low water solubility, that are not conducive to absorption by biological membranes. One technique that has been successful in improving dermal absorption of topically applied drugs is the creation of a physical eutectic system or complex with an excipient that has a lower melting point than the drug. Such systems provide much better dermal absorption, especially if the complex melting point is below body temperature and is lipophilic in nature. One such example is a eutectic system of 1:1 lidocaine base: prilocaine base termed ELMA which was introduced in 1993 and is among the leading anesthetic products (Friedman, 2001). Subsequent work indicated that formulation of the ELMA eutectic system into a micellar solution with a particle size <20 nm using POE-35-castor oil polymeric surfactant increased dermal permeation of the system by about six-fold (Fiala, 2016). The function of the excipient-drug active liquid complexes described herein is to mimic the absorption enhancing properties of the above-described eutectic systems.

Treatment Strategy Components

The above factors suggest that a dosage form having the following components should have increased potential for treatment of COVID-19. The latter would derive from mitigation of the adverse P/C properties of these drugs with regard to lung epithelial absorption and increase the ability of pulmonary administered CQ/HCQ to exert potential intracellular antiviral activity in addition to potential extracellular antiviral activity. The dosage form should also be easily sterilized through filtration and amenable to nebulization. The objective for such a dosage form is to provide rapid and effective epithelial absorption during the typically short residence times in the respiratory tract rather than the sustained release apparently provided by liposomal formulations.
An aminoquinoline liquid complex comprising the drug free base and a physiologically compatible excipient, ideally in a 1:1 mass ratio, that is liquid at body, and ideally ambient, temperature.
A polymeric surfactant suitable for administration to the respiratory tract that is capable of producing a micellar solution of the above aminoquinoline liquid complex at low surfactantliquid complex mass ratios (ideally 4:1 or lower) with micelle sizes less than 100 nm and ideally less than 50 nm.
An isotonic aqueous vehicle.
A nebulization device capable of producing aqueous droplet sizes for targeted delivery to various sites in the anatomical sites in the respiratory tract.

An exemplary formulation may have at least the following:
Saline USP selected as an isotonic aqueous vehicle.
CQ free base which has a reported melting point of 87° C.
CQ free base water-immiscible liquid complexes that are liquid at 20° C. have been prepared using 1:1 mass ratios with the following hydrophilic excipients: tetraethylene glycol, propylene glycol USP, 1,3-propanediol, 1,3-butylene glycol, and pentylene glycol. These are prepared by combining equal mass amounts of CQ free base and liquid excipient, heating to about 60° C. (well below melting point of CQ free base), and cooling to ambient temperature.
D-a-tocopheryl polyethylene glycol 1000 succinate NF (TPGS) may be the polymeric surfactant because it has been approved by the FDA as a safe adjuvant and widely used in drug delivery systems. The biological and physicochemical properties of TPGS provide multiple advantages for its applications in drug delivery like high biocompatibility, enhancement of drug solubility, improvement of drug permeation and selective antitumor activity. (Yang, 2018) TPGS has also been reported to enhance the immune response to diphitheria toxoid when administered intranasally. (Somavarapu, 2005) Its melting point is about 37-41° C. and it readily forms a homogenous melt with the above liquid complexes at a 4:1 TPGS: liquid complex mass ratio which will produce a micellar (visibly clear) solution in PBS at a 1 mg/mL CQ free base concentration (1 mg/mL liquid complex excipient and 8 mg/mL TPGS). POE-35-castor oil polymeric surfactant and block co-polymer surfactants such as poloxamer 127 may be suitable surfactants as well as mixtures of these three surfactants with each other and with other pharmaceutically acceptable surfactants. There are many potential surfactant candidates that could be used, but the present embodiments preferably use TPGS because of its profile and POE-35-castor oil and poloxamer 127 are suitable in similar applications for dermal delivery.
Successful micellar solutions have been prepared as described above using CQ liquid complexes with tetraethylene glycol, propylene glycol USP, and butylene glycol. Tetraethylene glycol has the same molecular structure as USP polyethylene glycols, but a considerably lower molecular weight, and has not as been extensively evaluated for safety, but can be considered as a preferred candidate as can propylene glycol, USP due to its extensive usage as a pharmaceutical excipient.

Alternate strategies are also provided within the scope of the present embodiments. According to one approach, a formulation strategy is to create a lipophilic ion pair with the charged drug species that is essentially water insoluble. This creates a “salt” that does not disassociate in water to form charged species and has appropriate properties for apical absorption. It could be administered in a micellar solution, but also as a molecular inclusion complex using modified cyclodextrins.

According to another approach, the drug uncharged species may be formulated into stable homogenous nano-sized (less than about 100 nm) aqueous dispersions through formation of inclusion complexes with modified cyclodextrins at about 1:1 to 1:2 molecular ratios and formation of water-soluble complexes with excipients such as 2-ethylhexanoic acid at mass ratios of about 1:1 to 1:2.

Other potential strategies to improve chloroquine topical bioavailability in the lung are feasible. The formulations are configured as a combination of pulmonary administration with a topical formulation that may be aerosolized designed to reduce/eliminate the putative issues affecting chloroquine topical bioavailability, namely is positive charge at physiological pH when administered as the diphosphate salt (primary form of chloroquine indicated in prior art), and the poor water solubility and very high lipophilicity of the chloroquine free base which is the species actually absorbed. One formulation strategy is thus to:
1. Use of chloroquine free base, and not the diphosphate salt.
2. Reduce lipophilicity of the free base through preparation of a “liquid complex”
3. Reduce impact of poor water solubility of free base or “liquid complex” through use of micellar solubilization with a suitable surfactant.

In another approach, the formulation may incorporate chloroquine free base in an inclusion complex in hydroxypropyl-s-cyclodextrin. This strategy uses chloroquine free base as the drug source and reduces the impact of the poor water solubility of the base through the use of a molecular inclusion complex of the free base. The latter solubilization technology may be advantageous as release from the inclusion complex may be superior to that from a swollen surfactant micelle. This may not apply to a “liquid complex” because of the size of the complex is presumed to be too large to incorporate into the hydrophobic interior of the cyclodextrin molecule. This inclusion complex may be formulated by a preparation by dissolving chloroquine free base in ethanol and addition the resulting solution to an aqueous solution of hydroxypropyl-β-cyclodextrin (could be in saline) such that the final concentration of ethanol is less than 5% and the molar ratio of hydroxypropyl-β-cyclodextrin to chloroquine base is from about 2:1 to 1:1.

In another approach, an additional strategy may involve the creation of a lipophilic ion pair of chloroquine base with an appropriate organic acid such that the resulting “salt” does not appreciably ionize in water such as the inorganic chloroquine salts (chloroquine diphosphate) do and therefore chloroquine is not presented to the absorbing epithelial membrane as a charged species; and has reduced lipophilicity relative to the free base and is better able to partition into the absorbing membrane.

Other approaches may include unmodified chloroquine free base formulated into transparent, homogenous aqueous dispersions through formation of molecular inclusion complex using hydroxypropyl-β-cyclodextrin at a 1:2 molar ratio or formation of a water-soluble complex with 2-ethylhexanoic acid at a 1:1 mass ratio. In yet another approach combinations of antiviral and antibiotic and/or anti-inflammatory treatments are considered. Catalysts may also be used to activate and deactivate composition components. According to one approach, the formulation may include a CQ free base with hydroxypropyl-R-cyclodextrin and 2-ethylhexanoic acid as described herein.

It should also be recognized that the principles for effecting pulmonary administration of approved drugs with antiviral activity may also be applied to additional moieties with therapeutic properties that may be additive or synergistic with those of the aforementioned drugs with antiviral activity. Such additional therapeutic moieties should be formulated in a manner so as to potentiate partitioning into and diffusion through apical membranes in the lung and to permit incorporation in the pulmonary dosage forms heretofore described. Examples of such moieties include, but are not limited to, lipophilic zinc compounds and antibiotic molecules.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention. Throughout this specification numerical labels of previously shown or discussed features may be reused to indicate similar features. Further, the terms atomization, nebulization and aerosolization may be used interchangeably to describe producing a fine spray, mist, minute particles, particle stream and/or colloidal suspension in the air.

References: The following references are incorporated herein in their entirety for all purposes.
Aderoumnu A F, Salako L A, Lindström B, Walker O, Ekman L, Comparison of the pharmacokinetics of chloroquine after single intravenous and intramuscular administration in healthy Africans, Br J clin Pharmac 22: 559 (1986)
American Hospital Formulary Service (AHFS) Drug Information, 2020
Clinical Trials Website, https://clinicaltrials.aov/ct2/show/study/NCT04477083; https://clinicaltrials.qov/ct2/show/NCT04461353
Devauz C A, Rolain J-M, Colson P, Raoult D, New insights on the antiviral effects of chloroquine against coronavirus: what to expect for COVID-19?, Int J Antimicrob Agents 55:105938 (2020)
Fasshihi S C, Nabar N R, Fassihi R, Novel approach for low-dose pulmonary delivery of hydroxychloroquine in COVID-19, Br J Pharmacol 177:4997 (2020)
Fiala S, Roman M, Inacio R, Mashal S, Brown M B, Jones S A, New insights into eutectic cream skin penetration enhancement, Int J Pharm 499:403 (2016)
Friedman P M, Mafong E A, Friedman E S, Geronemus R G, Topical anesthetics update: EMLA and beyond, Dematol Surg 27:1019 (2001)
Fruijtier-Polloth, Safety assessment on polyethylene glycols (PEGs) and their derivatives as used in cosmetic products, Toxicology 214:1 (2005)
Gardenhire, D.S. Rau's Respiratory Care Pharmacology. St. Louis: Elsevier, 2016.
Kaptein S J F, Jacobs S, Langendies L, Seldeslachts L, ter Horst S, Liesenborghs L, Hens B, Vergote V, Heylen E, Barthelemy K, Maas E, De Keyzer C, Bervoets L, Rymenants J, Van Buyten T, Zhang X, Abdelnabi R, Pang J, Williams R, Thibaut H J, Dallmeier K, Boudewijns R, Wouters J, Augustijns P, Verougstaete N, Cawthome C, Breuer J, Solas C, Weynand B, Annaert P, Spriet I, Vande Velde G, Neyts J, Rocha-Pereira J, Delang L, Favipiravir at high doses has potent antiviral activity in SARS-CoV-2-infected hamsters, whereas hydroxychloroquine lacks activity, PNAS 117:26955 (2020)
Kavanagh O, Healy A M, Dayton F, Robinson S, O'Reilly N J, Mahoney B, Arthur A, Walker G, Farragher J P, Inhaled hydroxychloroquine to improve efficacy and reduce harm in the treatment of COVID-19, Med Hypotheses 143:110110 (2020)
Klimke A, Hefner G, Wil B, Voss U, Hydroxychloroquine as an aerosol might markedly reduce and even prevent severe clinical symptoms after SARS-CoV-2 infection, Med Hypotheses 142:109783 (2020)
Labiris Nr, Dolovich M B, Pulmonary drug delivery. Part I: Physiological factors affecting therapeutic effectiveness of aerosolized medications, Br J Clin Pharmacol 56:588 (2003)
Liu J, Cao R, Xu M, Wang X, Zhang H, Hu, H Li Y, Hu Z, Zhnog W, Wang M, Hydroxychloroquine, a less toxic derivative of chloroquine, is effective in inhibiting SARS-CoV-2 infection in vitro, Cell Discovery 6:16 (2020)
Mayo Clinic Website, Mayo Clinic—Mayo Clinic
Osada N, Kohara A, Yamaji T, Hirayama N, Kasai F, Sekizuka T, Kuroda M, Handa K, The genome landscape of the African green monkey kidney-derived Vero Cell line, DNA Research 21: 673 (2014)
PubChem Website, PubChem (nih.qov)
Rosenke K, Jarvis M A, Feldmann F, Schwarz B, Okumura A, Lovaglio J, Saturday G, Hanley P W, Meade-White K, Williamson B N, Hansen F, Perez-Perez L, Leventhal S, Tang-Huau TpL, Nason M, Callison J, Haddock E, Scott D, Sewell G, Bosio C M, Hawman D, de Wit E, Feldmann, Hydroxychloroquine Proves Ineffective in Hamsters 1 and Macaques Infected with SARSCoV-2, JCI Insight. 5:e143174 (2020)
Somavarapu S, Pandit S, Gradassi G, Bandera M, Ravichandran E, Alpar O H, Effect of Vitamin E TPGS on immune response to nasally delivered diphtheria toxoid loaded poly(caprolactone) microparticles, Int J Pharm 298:244 (2005)
Si L, Bai H, Rodas M, Cao C, Oh C Y, Jiang A, Moller R, Hoagland D, Oishi K, Horiuchi S, Uhl S, Blanco-Melo D, Albrecht R A, Liu W-C, Jordan T, Nilsson-Payant B E, Logue J, Haupt R, McGrath M, Weston S, Nurani A, Kim S, Zhu D Y, Benam K H, Goyal G, Gilpin S E, Prantil-Baun R, Powers R K, Carlson K, Frieman M, tenOever BR, Ingber D E, Human organ chip-enabled pipeline to rapidly repurpose therapeutics during viral pandemics, bioRxiv https://doi.org/10.1101/2020.04.13.039917 Posted Aug. 20, 2020
Tai T-T, Wu T-J, Wu H-D, Tsai Y-C, Wang H-T, Wang A-M, S S-F, Chen Y-C, A strategy to treat COVID-19 disease with targeted delivery of inhalable liposomal hydroxychloroquine: a preclinical pharmacokinetic study, Clin Transl Sci 0:1 (2020)
Tett S E, Cutler D J, Day R O, Brown K F, A dose-ranging study of the pharmacokinetics of hydroxychloroquine following intravenous administration to healthy volunteers, Br J clin Pharmac 26: 303 (1988)
US Food and Drug Administration (US FDA) “Drug Safety and Availability” (US FDA,2020); https://www.fda.gov/drugs/drug-safety-and-availability/fda-cautions-aqainst-use-hydroxvchloroquine-or-chloroquine-covid-19-outside-hospital-settinq-or
Wang M, Cao R, Zhang L, Yang X, Liu J, Xu M, Shi Z, Hu Z, Zhong W, Xiao G, Remdesivir and chloroquine effectively inhibit the recently emerged novel coronavirus (2019-nCoV) in vitro, Cell Research 30: 269 (2020)
White N J, Looareesuwan S, Edwards G, Phillips R E, Karbwang J, Nicholl D D, Bunch C, Warrell D A, Pharmacokinetics of intravenous amodiaquine, Br J clin Pharmac 23: 127 (1987)
World Health Organization (WHO). “Solidarity” clinical trial for COVID-19 treatments. (WHO, 2020); https://www.who.intiemergencies/diseases/novel-coronavirus-2019/qlobal-research-on-novel-coronavirus-2019-ncov/solidarity-clinical-trial-for-covid-19-treatments
Yang C, Wu T, Qi Y, Zhang Z, Recent Advances in the Application of Vitamin E TPGS for Drug Delivery, Theranostics 8:464 (2018)
Yao X, Ye F, Zhang M, Cui C, Huang B, Niu P, Liu X, Zhao L, Dong E, Song C, Zhan S, Lu R, Li H, Tan W, Liu D, In Vitro Antiviral Activity and Projection of Optimized Dosing Design of Hydroxychloroquine for the Treatment of Severe Acute Respiratory Syndrome Coronavirus 2 (SARS-CoV-2), Clin Infect Dis 71: 732 (2020)
Zhou Q, Leung S S Y, Tang P, Parumasivam T, Loh Z H, Chan H-K, Inhaled formulations and pulmonary drug delivery systems for respiratory infections, Adv Drug Delivery Rev 85:83 (2015)
Alvarez-Trujillo L A, Lazic V, Laserna J J, Standoff monitoring of aqueous aerosols using nanosecond laser-induced breakdown spectroscopy: droplet size and matrix effects, Appl Opt. 2017 May 1;56(13):3773-3782, DOI: 10.1364/AO.56.003773
Kooij S, sijs R, Denn M M, Villermauz E, Bonn D, Mat determines the Drop Size in Sprays?, Physical Review X 8, 031019 (2018), DOI: 10.1103/PhysREvX.8.031019

### REFERENCE NUMBERS

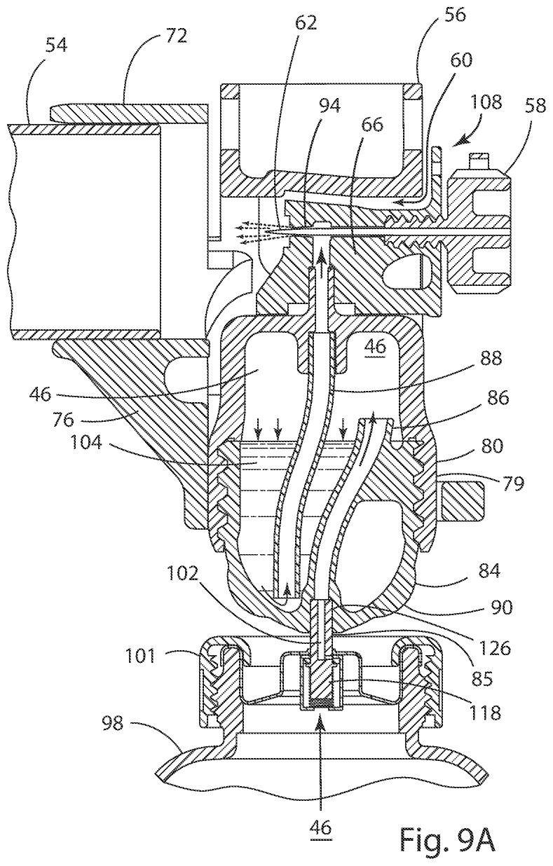

20 Exemplary embodiment of a nebulizer
21 main body housing
22 rmouth piece input opening
23 medication
24 output mouth piece
23 twist valve chamber seal
26 twist valve chamber
27 aerosolized/medication
28 medication chamber
29 side pipe
30 nebulization chamber
31 nebulized exit point
32 jet to nebulize medication
33 final nebulized exit point
34 handle
36 air chamber
38 piston
40 spring
42 lock lever to hold tensioned spring
44 pressure release valve
46 air or other medically approved gas
50 Exemplary embodiment of a nebulizer according to another approach
52 facemask
54 aerosol chamber
56 living hinge/nozzle housing interface
58 droplet size control knob
60 droplet size control knob thread
62 nebulizer needle
64 droplet size presets
66 nebulizer housing
70 medication chamber latch arm lock
72 face mask/housing interface
74 living hinge/frame interlaces
76 nebulizer frame
78 nebulizer frame stop
79 upper medication chamber
80 upper medication chamber cap
82 upper medication chamber interface to nebulizer frame
84 lower medication chamber
85 lower medication chamber compressed air chamber interface
86 compressed air channel
87 connector for lower medication chamber 84 and upper medication chamber cap 80
88 aerosol channel
90 lower medication chamber ergonomic indentations
92 actuation arms
94 nebulizer housing orifice
96 actuation arm lock
97 actuation arm lock
98 compressed air chamber
99 hinge point
100 hinge pint
102 valve 118 hollow stem
104 medication
106 compressed air port (e.g., valve stem)
108 ambient air channel (FIG. 8)
110 paul to guide wings 96 and 97 of the actuation arm lock
111 alignment arrows
112 rib
114 rib
116 track
118 air chamber/lower medication chamber interface spring loaded valve
120 grooves
122 fins
124 stop
126 bore to receive stem 102
208 human respiratory anatomy
210 nose
212 mouth
214 lower respiratory portion
216 lung periphery portion
218 nasal cavity
219 oral cavity 119
220 pharynx
222 larynx 122
224 trachea 24
226 lungs 126
228 bronchi

What is claimed is:

1. An inhalation formulation, comprising:
an aerosolizable formulation having
a pharmaceutically active anti-viral compound present as a neutral compound selected from the group of free acid, free base, water insoluble salt and water insoluble ion pair);
an excipient capable of forming a liquid complex with the pharmaceutically active anti-viral compound; and
a polymeric surfactant suitable for pulmonary administration.

2. The formulation of claim 1, wherein the pharmaceutically active anti-viral compound is at least one of: chloroquine (CQ), hydroxychloroquine (HCQ) and amodiaquine.

3. The formulation of claim 1 wherein the excipient forming a liquid complex with the pharmaceutically active anti-viral compound is present in a 0.2:1 to a 5:1 excipient to drug mass ratio.

4. The formulation of claim 3 in which the excipient forming a liquid complex with the pharmaceutically active anti-viral compound is present in a 1:1 excipient to drug mass ratio.

5. The formulation of claim 3 in which the excipient is propylene glycol, USP.

6. The formulation of claim 1, wherein the polymeric surfactant suitable for administration to the respiratory tract is capable of producing a micellar solution with the drug liquid complex at mass ratios to the drug liquid complex of 8:1 or lower.

7. The formulation of claim 6, wherein the polymeric surfactant suitable for administration to the respiratory tract is capable of producing a micellar solution with the drug liquid complex is tocopheryl polyglycerol succinate.

8. The formulation of claim 1, wherein aerosolized formulation comprises micelle sizes less than 100 nm.

9. The formulation of claim 1, wherein the aerosolized droplets are less than 5 μm and lipoidal particles containing approved drugs are less than 100 nm.

10. A nebulizer suitable for medication delivery to the lungs, comprising:
a compressed air chamber in communication with a medication chamber, the communication sealed by a spring valve in a rested state, the spring valve being openable in an actuated state,
a nebulizer chamber in communication with the medication chamber, the nebulizer chamber having a pressure release orifice,
a facemask integral or in direct communication with the nebulizer chamber, wherein the nebulizer chamber is configured to deliver a stream the nebulized particle stream to the facemask,
wherein the compressed air chamber is configured to have the volume and pressure of air needed to nebulize the medication through the pressure release orifice,
wherein the nebulizer chamber has a continuously variable nebulizing pressure feed at the pressure release orifice; and
wherein the spring valve is released by a pair of levers that when actuated, force the medication chamber and the gas chamber together to open the spring valve.

11. The nebulizer of claim 10, wherein the continuously variable nebulizing pressure feed has a retractable and extendable needle to retract and extend into the pressure release orifice.

12. The nebulizer of claim 10, wherein the retractable needle is attached to a threaded shaft disposed within a threaded bore, which is rotatable by a control knob to retract and extend into the pressure release orifice.

13. The nebulizer of claim 10, wherein nebulizer chamber is configured to produce a nebulized particle stream in the range of 1-10 μm.

14. The nebulizer of claim 10, wherein the medication is an antiviral with a carrier, which is nebulizable to a particle stream in the range of 3-5 μm.

15. The nebulizer of claim 10, wherein the force required to open the spring valve to an actuated state is less than 3 nM of force.

16. The nebulizer of claim 10, wherein the actuation levers remain aligned along its travel path by a guide track on one of the levers and a paul, which is guided within the track, on the other lever.

17. The nebulizer of claim 10, wherein the compressed air chamber is configured to hold up to 120 PSI of air.

18. The nebulizer of claim 10, wherein the compressed air chamber is configured to hold in the range of 20-60 PSI of air.

19. The nebulizer of claim 10, wherein the pressure release orifice is configured to produce aerosolized fluid of varying droplet/particle sizes ranging from 1-10 micrometers in diameter.

20. The nebulizer of claim 10, wherein the pressure release orifice is in the range of 0.35-2.00 mm in diameter.
